(19) United States
(12) Patent Application Publication
(10) Pub. No.: US 2024/0116879 A1
(43) Pub. Date: Apr. 11, 2024

(54) AMILORIDE DERIVATIVES AND METHODS OF USING SAME FOR THE TREATMENT OF VIRAL INFECTIONS

(71) Applicants: Duke University, Durham, NC (US); Case Western Reserve University, Cleveland, OH (US); Rutgers, The State University of New Jersey, New Brunswick, NJ (US)

(72) Inventors: Amanda Hargrove, Durham, NC (US); Blanton Tolbert, Cleveland, OH (US); Gary Brewer, New Brunswick, NJ (US); Neeraj Narendra Patwardhan, Durham, NC (US); Mei-Ling Li, New Brunswick, NJ (US)

(73) Assignees: Duke University, Durham, NC (US); Case Western Reserve University, Cleveland, OH (US); Rutgers, The State University of New Jersey, New Brunswick, NJ (US)

(21) Appl. No.: 18/039,917

(22) PCT Filed: Dec. 3, 2021
(86) PCT No.: PCT/US21/61774

§ 371 Date: Jun. 1, 2023

Related U.S. Application Data
(60) Provisional application No. 63/120,912, filed on Dec. 3, 2020.

(51) Int. Cl. C07D 241/34; A61P 31/14

(52) U.S. Cl. CPC C07D 241/34; A61P 31/14

(57) ABSTRACT

The present disclosure describes, in part, compositions comprising derivatives and methods of using the same in prevention or treatment of viral infections in a subject.

### CROSS REFERENCE TO RELATED APPLICATIONS

This PCT application claims benefit of priority to U.S. Provisional Patent Application No. 63/120,912, filed Dec. 3, 2020, the contents of which is hereby incorporated by reference in its entirety.

### FEDERAL FUNDING LEGEND

This invention was made with Government support under Federal Grant Nos. R35 GM124785 and R01 GM126833 awarded by the National Institutes of Health (NIH). The Federal Government has certain rights to this invention.

### BACKGROUND

Severe Acute Respiratory Syndrome Coronavirus 2 (SARS-CoV-2) is the etiological agent of the COVID-19 respiratory disease, the largest scale respiratory virus pandemic the world has witnessed since the 1918 Spanish flu. Coronaviruses (CoVs) generally cause mild flu-like symptoms in humans but have caused two smaller scale pandemics in the last two decades: SARS-CoV (2003) and MERS (2012) (Costa et al., Arch Virol 165, 1517-1526 (2020)). Recent phylogenetic mapping traced all human coronaviruses to animal origins (Cui et al., Nat Rev Microbiol 17, 181-192 (2019)). While the middle zoonotic carrier of the virus between the animal of origin and humans seem to vary between CoVs, the chronological surfacing of human CoV pandemics seems to follow a dangerous trend of increasing lethality of each pandemic, thereby underscoring the need for a better understanding and targeting of the current and future CoV etiologic agents.

After more than a year since the first cases of SARS-CoV-2 human infection, this virus is expected to remain a global threat until vaccines are available and adopted worldwide. Furthermore, despite the increase in number of approved vaccines their implementation has been hindered by the scarcity of doses available worldwide. On top of production rate, pharmaceutical companies are currently facing challenges with the recently reported SARS-CoV-2 variants, against which not all vaccines have proven sustained efficacy, highlighting the need for a synergistic antiviral-based approach (Greaney et al., Cell Host & Microbe 29, 463-476.e466 (2021)). While recent treatments have been approved for use within hospital settings, there are no known FDA approved cures for the infection (Release, FDA news (2020)).

The SARS-CoV-2 pandemic, and the likelihood of future coronavirus pandemics, emphasized the urgent need for development of novel antivirals. Small molecule chemical probes offer to both reveal novel aspects of virus replication and to serve as leads for antiviral therapeutic development.

### SUMMARY

The present disclosure is based, in part, on results of analysis of the CoV RNA genome that revealed many conserved RNA structures in the 5′-end region critical for viral translation and replication, including several containing bulge-like secondary structures suitable for small molecule targeting. Following conservation analysis of this region, a small molecule library based on the amiloride scaffold was screened against a less virulent human coronavirus, OC43, to identify lead ligands. Next, antiviral activity was replicated in SARS-CoV-2 via a rapid RT-q-PCR based assay and authentic viral infections. Reporter screens confirmed the importance of RNA structures in the 5′ end of the viral genome. Finally, NMR chemical shift perturbations studies of the first six stem loops of the 5′-end revealed specific amiloride interactions with stem loops 4, 5a, and 6, all of which contain internal or bulge like structures and were predicted to be strongly bound by the lead amilorides in retrospective docking studies. This combination of methods has revealed the first small molecules to target RNA structures at the 5′-end of the CoV genome. These molecules serve as chemical probes to further understand coronavirus RNA biology, and the combination of methods allows rapid identification of future RNA-targeted antivirals.

Accordingly, one aspect of the present disclosure provides a compound of Formula (I), a pharmaceutical acceptable salt, a hydrate, a prodrug, an ester, or a derivative thereof:

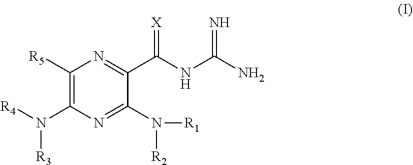

wherein R₁ and R₂ are independently H, C₁-C₈ alkyl, substituted C₁-C₈ alkyl, C₁-C₈ alkenyl, substituted C₁-C₈ alkenyl, C₃-C₆ substituted cycloalkyl, a C₃-C₆ unsubstituted cycloalkyl, aryl, or substituted aryl;
R₃ and R₄ are independently H, C₁-C₈ alkyl, substituted C₁-C₈ alkyl, C₁-C₈ alkenyl, substituted C₁-C₈ alkenyl, C₃-C₆ substituted cycloalkyl, a C₃-C₆ unsubstituted cycloalkyl, aryl, substituted aryl, or R³ and R⁴ may be taken together to form a C₃-C₆ substituted cycloalkyl, a C₃-C₆ unsubstituted cycloalkyl, C₃-C₆ substituted cycloalkenyl, a C₃-C₆ unsubstituted cycloalkenyl, a substituted heterocycle, a unsubstituted heterocycle, or a C₃-C₂₄ heteroaromatic;
R₅ is C₁-C₈ substituted alkyl, C₁-C₈ unsubstituted alkyl, C₃-C₆ substituted cycloalkyl, C₃-C₆ unsubstituted cycloalkyl, a substituted heterocycle, an unsubstituted heterocycle, C₃-C₂₄ aromatic, C₃-C₂₄ heteroaromatic,

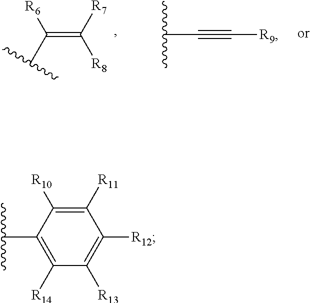

and
R₆, R₇, R₈, R₉, R₁₀, R₁₁, R₁₂, R₁₃, and R₁₄ independently are H, Cl, Br, F, CF₃, C₁-C₆ substituted alkyl, C₁-C₆ unsubstituted substituted alkyl, OCH₃, NH₂, NHCH₃, N(CH₃)₂, CN, cyclopropyl, cyclobutyl, cyclopentyl, cyclohexyl, or phenyl; and X is O, S, or NH.

In some embodiments, R₁ and R₂ may be independently H, C₁-C₄ alkyl, C₁-C₆ substituted alkyl, C₁-C₄ alkenyl, C₃-C₅ substituted cycloalkyl, a C₃-C₅ unsubstituted cycloalkyl, aryl, or substituted aryl; R₃ and R₄ may be independently H, C₁-C₄ alkyl, C₁-C₄ substituted alkyl, C₁-C₄ alkenyl, C₃-C₅ substituted cycloalkyl, a C₃-C₅ unsubstituted cycloalkyl, aryl, substituted aryl, or R³ and R⁴ may be taken together to form a C₃-C₅ substituted cycloalkyl, a C₃-C₅ unsubstituted cycloalkyl, C₄-C₁₄ heteroaromatic, a substituted heterocycle, or an unsubstituted heterocycle; R₅ may be C₁-C₄ substituted alkyl, C₁-C₄ unsubstituted alkyl, C₃-C₅ substituted cycloalkyl, C₃-C₅ unsubstituted cycloalkyl, C₄-C₁₄ aromatic, C₄-C₁₄ heteroaromatic, a substituted heterocycle, an unsubstituted heterocycle,

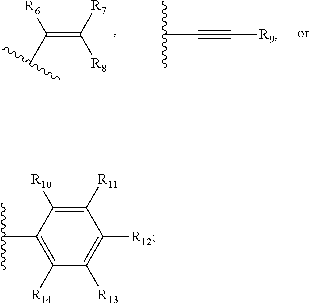

R₆, R₇, R₈, R₉, R₁₀, R₁₁, R₁₂, R₁₃, and R₁₄ may be independently H, Cl, Br, F, OCH₃, NH₂, NHCH₃, N(CH₃)₂, or phenyl; and X may be O.

In some embodiments, R₁, R₂, R₃, and R₄ may be independently H, CH₃, CH₃CH₂,, CH(CH₃)₂, C(CH₃)₃, or phenyl; R₅ may be

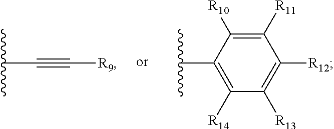

R₆, R₇, R₈, R₉, R₁₀, R₁₁, R₁₂, R₁₃, and R₁₄ independently may be H, CH₃, F, CF₃, or phenyl; and X may be O.

In some embodiments, R₁, R₂, R₃, and R₄ may be independently H or CH₃; R₅ is,

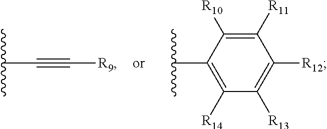

R₉, R₁₀, R₁₁, R₁₂, R₁₃, and R₁₄ independently may be H or phenyl; and X may be O.

In some embodiments, the compound may be DMA-132, wherein R₁ and R₂ are H; R₃, and R₄ are CH₃;

R₅ is

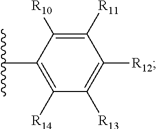

and R₁₀, R₁₁, R₁₂, R₁₃, and R₁₄ are H as shown in the compound of Formula (II):

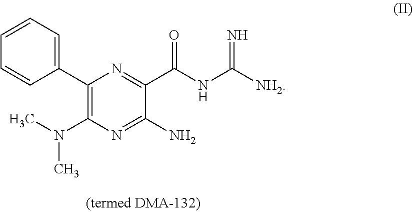 [SMILES: CN(C)c1nc(N)c(C(=O)NC(=N)N)nc1-c1ccccc1]

In some embodiments, the compound may be DMA-135, wherein R₁ and R₂ are H; R₃, and R₄ are CH₃:

R₅ is

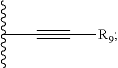

and R₉ is phenyl as shown in the compound of Formula (III):

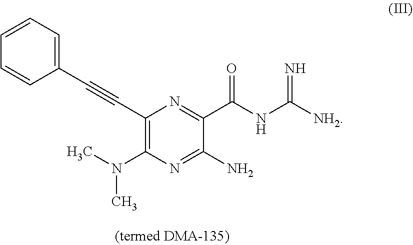 [SMILES: CN(C)c1nc(N)c(C(=O)NC(=N)N)nc1C#Cc1ccccc1]

In some embodiments, the compound may be DMA-155, wherein R₁ and R₂ are H; R₃, and R₄ are CH₃;

R₅ is

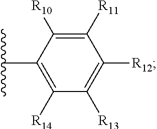

and R₁₀, R₁₁, R₁₃, and R₁₄ are H; R₁₂ is phenyl as shown in the compound of Formula (IV):

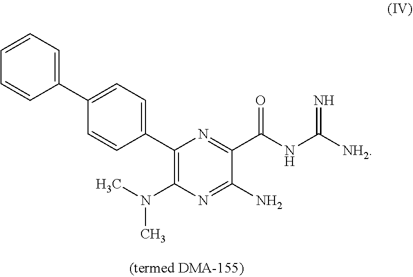 [SMILES: CN(C)c1nc(N)c(C(=O)NC(=N)N)nc1-c1ccc(-c2ccccc2)cc1]

Another aspect of the present disclosure provides a pharmaceutical composition comprising, consisting of, or consisting essentially of any of the compounds as described above and herein and a pharmaceutically acceptable carrier and/or excipient.

Another aspect of the present disclosure provides a method of inhibiting viral replication in a cell. This method may comprise administering to the cell an effective amount of any of the compounds or an effective amount of the pharmaceutical composition as described above and herein.

Another aspect of the present disclosure provides a method of preventing or treating a viral infection in a subject. This method may comprise administering to the subject an effective amount of any of the compounds or an effective amount of the pharmaceutical composition as described above and herein.

In some embodiments, the virus may comprise a coronavirus. In some embodiments, the coronavirus may be selected from the group consisting of 229E (alpha coronavirus), NL63 (alpha coronavirus), OC43 (beta coronavirus), HKU1 (beta coronavirus), MERS-CoV (beta coronavirus), SARS-CoV (beta coronavirus), and SARS-CoV-2, or a combination thereof. In certain embodiments, the coronavirus may comprise SARS-CoV-2.

Still another aspect of the present disclosure provides a method of preventing or treating COVID-19 in a subject. This method may comprise administering to the subject an effective amount of any of the compounds or an effective amount of the pharmaceutical composition as described above and herein.

In some embodiments, for any of the methods described above and herein, the compound or the pharmaceutical composition may be administered in conjunction with another antiviral or anti-inflammatory agent. In certain embodiments, the compound or the pharmaceutical composition and another antiviral or anti-inflammatory agent may be combined in the same formulation or pill.

Still another aspect of the present disclosure provides use of any of the compounds or the pharmaceutical composition described above and herein as an antiviral agent.

In some embodiments, the antiviral agent targets coronavirus RNA. By way of non-limiting example, the coronavirus may comprise 229E (alpha coronavirus), NL63 (alpha coronavirus), OC43 (beta coronavirus), HKU1 (beta coronavirus), MERS-CoV (beta coronavirus), SARS-CoV (beta coronavirus), SARS-CoV-2, or a combination thereof. In certain embodiments, the coronavirus may comprise SARS-CoV-2.

Yet another aspect of the present disclosure provides use of any of the compounds or the pharmaceutical composition described above and herein as an antiviral agent for preventing or treating COVID-19.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying figures and examples are provided by way of illustration and not by way of limitation. The foregoing aspects and other features of the disclosure are explained in the following description, taken in connection with the accompanying figures and examples relating to one or more embodiments, in which.

FIG. 1 is a schematic showing SARS-CoV-2 replication cycle. The virus enters human cells via endocytosis by binding the ACE2 receptor and releasing its positive-sense RNA genome. The virus exploits the host machinery to facilitate efficient viral replication, which ultimately leads to progression of infection.

FIG. 2 is a schematic showing the secondary structure representation of the 5′-end (450 nt) of the SARS-CoV-2 RNA genome as determined by dimethyl sulfate (DMS) chemical probing. Structure recreated in VARNA.

FIG. 3 shows the conservation of 5′-end sequences encoding RNA structures in betacoronavirus genomes. Multiple sequence alignment showing percentage identity and sequence coverage within the 5′ untranslated and adjacent region of representative species within genus Betacoronavirus. Viral subgenus is indicated as shown in the key. Sequence numbering is standardized to the genome sequence start position of each taxon. Taxon labels show GenBank accession numbers and abbreviated virus names. Abbreviations: OC43=Human coronavirus OC43; SARS=Severe acute respiratory syndrome coronavirus; RousettusBat=Rousettus bat coronavirus MERS=Middle East respiratory syndrome-related coronavirus; Bat-Hibeco=Bat Hp-betacoronavirus.

FIG. 4 shows small molecule screening against OC43 with Vero E6 cells. Vero E6 cells were infected with human coronavirus OC43 at an MOI=1. Various concentrations of dimethylamilorides (DMAs) were added to the cells. Media were harvested 24 hr post-infection and assayed for infectious virus by plaque formation with Vero E6 cells.

FIG. 5 shows the chemical structures of the three lead molecules derived from the focused library screen against OC43 infected Vero E6 cell screening.

FIG. 6 shows replicates of antiviral effects of lead DMAs. Vero E6 cells were infected with human coronavirus OC43 at an MOI=1. DMAs were added to the cells at 50 μM or 100 μM. Media were harvested 24 hr post-infection and assayed for infectious virus by plaque formation with Vero E6 cells. Mean values and standard deviations from three independent experiments are shown in the bar graphs. P<0.01 for all three DMAs.

FIGS. 7A-7B show Q-RT-PCR assay for dose dependent antiviral activity of various DMAs (FIGS. 7A-7C: DMA-1; FIGS. 7D-7F: DMA-132; FIGS. 7G-7I: DMA-135; FIGS. 7J-7L: DMA-155) and Remdesivir (FIGS. 7M-7O). Vero E6 cells were infected with SARS-CoV-2 at MOI: 0.1 i.u./cell and the indicated DMA compounds were added on cells following virus adsorption for 1 hr. Cell culture supernatants were collected and analyzed by a Q-RT-PCR assay using N-specific primers. Data show the relative percentage of viral RNA in DMA-treated samples compared to mock-treated samples at 24, 48, and 72 hours. Data are from two independent experiments and error bars show the range.

FIG. 8 is a graph showing inhibition of SARS-CoV-2 infection by DMA leads without significant cellular toxicity. VeroE6 cells were infected with SARS-CoV-2 (MOI=0.1) in the presence of DMSO or the indicated compounds for 3 days followed by normalized measurement of viral titer in culture supernatant (black bars). Results were generated with two independent experiments, each with two replicates. Cellular viability was assayed by measuring cellular ATP content in uninfected VeroE6 cells after 3 days of treatment with the indicated compounds (grey bars).

FIGS. 9A-9C show small molecule toxicity in Vero E6 cells. Cytotoxicity assay shows the CC₅₀ of DMA-132 (FIG. 9A) and DMA-135 (FIG. 9B) in Vero E6 cells were>100 μM. CC₅₀ of DMA-155 was about 90 μM (FIG. 9C). Various concentrations of DMAs were added to Vero E6 cells. Cells were incubated at 33° C. for 96 hrs. Cell viability was determined by MTT assay with measurements at 570 nm according to the manufacturer's instructions (EMD Millipore). All experiments were performed in triplicate.

FIGS. 10A-10B illustrate Vero E6 cells co-transfected with luciferase constructs and cultured with various concentration of small molecule. Vero E6 cells were co-transfected with CoV-2-5′UTR-FLuc-3′UTR (FIG. 10A). Vero E6 cells were co-transfected with Cov-2-5′UTR-FLuc (left) and no change in inhibition was observed when compared to the presence of 3′-UTR (right, DMA-135) (FIG. 10B). Luciferase activity was measured 2 days later. Mean values and standard deviations from three independent experiments are shown in the bar graphs. ***P<0.001; **P<0.01; N., not significant relative to the DMSO control, except for the 5′UTR-FLuc-3′UTR comparison in panel FIG. 10B, which is relative to DMA-135 at 0.1 μM.

FIGS. 11A-11D show that single-point ¹³C-¹H TROSY HSQC titrations reveal DMA-132 -135 and -155 bind with moderate affinity and specificity to SARS-CoV-2 5′-region stem loops. (A(¹³C, ¹⁵N) SL1—FIG. 11A; A(¹³C, ¹⁵N) SL4—FIG. 11B; AU(¹³C, ¹⁵N) SLSA—FIG. 11C; AC(¹³C, ¹⁵N) SL6—FIG. 11D). The spectra were recorded at 900 MHz in 100% D₂O buffer of 25 mM K₂HPO₄, 50 mM KCl at pH 6.2. Temperatures (298, 303 or 308 K) were optimized for each RNA construct to maximize the number of observed correlation peaks. The total RNA concentrations were set to 100 μIM while titrating 5-fold excess DMA.

FIGS. 12A-12D show ¹³C-HSQC NMR experiments SARS-CoV2 5′-end RNA structures with DMAs. Single-point ¹³C-¹H TROSY HSQC titrations of DMA-132, DMA-135 and DMA-155 with isolated SL domains of the SARS-CoV-2 5′-end (C(¹³C, ¹⁵N) SL1—FIG. 12A; U(¹³C, ¹⁵N) SL2—FIG. 12B; A(¹³C, ¹⁵N) SL3—FIG. 12C; AU(¹³C, ¹⁵N) SLSB—FIG. 12D).

FIGS. 13A-13C show ¹H-¹H NOESY NMR experiments of selectively labeled SL1 with DMA-135. DMA-135 changes local structure of the SARS-Cov-2 5′-SL1 domain, while DMA-155 preserves it. (FIG. 13A) Overlay of ¹H-¹H NOESY spectra (tₘ=250 ms) of free GC(²H), AU(²H_{3′-5″})-selectively labeled SL1 (blue) and the (DMA-135)-SL1 complex (red). (FIG. 13B) ¹H-¹H NOESY spectra overlay (tₘ=250 ms) of free GC(²H), AU(² H_{3′-5″})-selectively labeled SL1 (blue) and the (DMA-155)-SL1 complex (red). All NMR spectra were collected at 900 MHz in 25 mM K2HPO4, 50 mM KCl, pH 6.2 D2O buffer at 303 K. The selectively labeled nucleotides are highlighted in red on the secondary structure of SL1 (FIG. 13C).

FIGS. 14A-14C show detailed interaction of DMA-155 with stem loop 6 (SL6). Representation of SL6 secondary structure and labeled nucleotides (red) (FIG. 14A). ¹H-¹H NOESY spectra (900 MHz, tₘ=250 ms) of free UG(²H), AC(²H_{3′-5′})-selectively labeled SL6 (blue) and its DMA-155 complex (red), which were collected in 25 mM K₂HPO₄, 50 mM KCl pH 6.2 at 308K in 100% D2O, show that DMA-155 has a degree of binding specificity (FIG. 14B). ¹H-¹H NOESY spectra (900 MHz and tₘ=250 ms) of free CUG(²H), A(²H_{3′-5″)}-selectively labeled SL6 (red), its DMA-155 complex (black), and the fully deuterated RNA complexed with DMA-155 (orange) were collected in 25 mM K₂HPO₄, 50 mM KCl pH 6.2 at 308K in 100% D₂O (FIG. 14C). On the secondary structure of SL6, the nucleotides highlighted in red represent the labeling scheme of the RNA.

FIGS. 15A-15F show that retrospective docking substantiates NMR findings. FIG. 15A shows secondary structure representation of SL1 construct used in NMR studies. FIG. 15B shows SL1 3D model with binding pockets highlighted in different colors identified via ICM pocket finder. The model depicted is a representative conformer of the 15-confomers cluster generated for the internal loop motif. FIG. 15C shows conformation and binding pockets that yielded the best docking scores of the three hit molecules DMA 132 (red); DMA 135 (green), and DMA 155 (purple). FIG. 15D shows secondary structure representation of SL6 construct used in NMR studies. FIG. 15E shows SL6 3D model with binding pockets (red and blue) identified via ICM pocket finder. Model depicted is a representative conformer of the 15-structure clusters built for internal loop motif. FIG. 15F shows conformer that yielded the best docking scores for the three hit molecules, namely DMA 132 (red), DMA 135 (green), and DMA 155 (purple) represented in space filling model.

FIG. 16 shows docking score of the 55 member DMA library against stem loop 1 (SL1). The dots represent the best docking scores for each SL1 conformer present in the cluster of 10 structures.

FIG. 17 shows docking score of the 55 member DMA library against stem loop 3 (SL3). The dots represent the best docking scores for each SL3 conformer present in the cluster.

FIG. 18 shows docking score of the 55 member DMA library against stem loop 4 (SL4). The dots represent the best docking scores for each SL4 conformer present in the cluster.

FIG. 19 shows docking score of the 55 member DMA library against stem loop 5a (SLSa). The dots represent the best docking scores for each SLSa conformer present in the cluster.

FIG. 20 docking score of the 55 member DMA library against stem loop 5b (SLSb). The dots represent the best docking scores for each SLSb conformer present in the cluster.

FIG. 21 shows docking score of the 55 member DMA library against stem loop 6 (SL6). The dots represent the best docking scores for each SL6 conformer present in the cluster.

FIG. 22 shows refined docking on motif-based clusters. Docking score of DMA-132 against every stem loop. Columns represented correspond to pockets identified in bulge regions of motifs created around bulge or internal loop motifs. The dots correspond to the docking score of the molecule against each of the 15 conformers within the cluster.

FIG. 23 shows refined docking on motif-based clusters. Docking score of DMA-135 against every stem loop. Columns represented correspond to pockets identified in bulge regions of motifs created around bulge or internal loop motifs. The dots correspond to the docking score of the molecule against each of the 15 conformers within the cluster.

FIG. 24 shows refined docking on motif-based clusters. Docking score of DMA-155 against every stem loop. Columns represented correspond to pockets identified in bulge regions of motifs created around bulge or internal loop motifs. The dots correspond to the docking score of the molecule against each of the 15 conformers within the cluster.

FIG. 25A shows small molecule titration curve of DMA-135 with SL1, SL4, SL5a, SL5b, and SL6. Small molecule binding is plotted as a function % displacement of a fluorescent dye and ligand concentration. FIG. 25B shows affinity calculated for DMA-135 for each stem loop after three independent replicates.

FIG. 26A shows small molecule titration curve of DMA-155 with SL1, SL4, SL5a, SL5b, and SL6. Small molecule binding is plotted as a function % displacement of a fluorescent dye and ligand concentration. FIG. 26B shows Affinity calculated for DMA-155 for each stem loop after three independent replicates.

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

As used herein, the term “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. As used herein, the terms “prevent,” “preventing,” “prevention,” “prophylactic treatment” and the like refer to reducing the probability of developing a disease, disorder or condition in a subject, who does not have, but is at risk of or susceptible to developing a disease, disorder or condition. The term “effective amount” or “therapeutically effective amount” refers to an amount sufficient to effect beneficial or desirable biological and/or clinical results.

As used herein, the term “administering” an agent, such as a therapeutic entity to an animal or cell, is intended to refer to dispensing, delivering or applying the substance to the intended target. In terms of the therapeutic agent, the term “administering” is intended to refer to contacting or dispensing, delivering or applying the therapeutic agent to a subject by any suitable route for delivery of the therapeutic agent to the desired location in the animal, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

The term “biological sample” as used herein includes, but is not limited to, a sample containing tissues, cells, and/or biological fluids isolated from a subject. Examples of biological samples include, but are not limited to, tissues, cells, biopsies, blood, lymph, serum, plasma, urine, saliva, mucus and tears. In one embodiment, the biological sample is a biopsy (such as a tumor biopsy). A biological sample may be obtained directly from a subject (e.g., by blood or tissue sampling) or from a third party (e.g., received from an intermediary, such as a healthcare provider or lab technician).

The term “disease” as used herein includes, but is not limited to, any abnormal condition and/or disorder of a structure or a function that affects a part of an organism. It may be caused by an external factor, such as an infectious disease, or by internal dysfunctions, such as cancer, cancer metastasis, and the like.

“Contacting” as used herein, e.g., as in “contacting a sample” refers to contacting a sample directly or indirectly in vitro, ex vivo, or in vivo (i.e. within a subject as defined herein). Contacting a sample may include addition of a compound to a sample (e.g., a sample comprising cells infected with a viral infection), or administration to a subject. Contacting encompasses administration to a solution, cell, tissue, mammal, subject, patient, or human. Further, contacting a cell includes adding an agent to a cell culture.

As used herein, the term “viral infection” refers to those conditions characterized by the presence of a virus in the body (of a subject). The subject may show symptoms (e.g., fever, chills, stuffy/runny nose, etc.) or be asymptomatic. The methods and formulations provided herein are also useful in the prevention of a viral infection and may be used as a prophylactic for a subject at risk of developing a viral infection (e.g., a medical professional). The viral infection may be caused by any kind of virus. In some embodiments, the virus is capable of infecting a human subject. In some embodiments the virus is capable of infecting the mucosal/nasal/lungs/respiratory tract. Suitable examples include influenza, coronaviruses, rhinoviruses, respiratory syncytial virus, and the like. In some embodiments, the viral infection may be caused by a coronavirus. In some embodiments, the coronavirus may be selected from the group consisting of 229E (alpha coronavirus), NL63 (alpha coronavirus), OC43 (beta coronavirus), HKU1 (beta coronavirus), MERS-CoV (beta coronavirus), SARS-CoV (beta coronavirus), and SARS-CoV-2, or a combination thereof. By way of non-limiting example, the coronavirus may comprise SARS-CoV-2.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e. living organism, such as a patient).

Unless otherwise defined, all technical terms used herein have the same meaning as commonly understood by one of ordinary skill in the art to which this disclosure belongs.

### A. Compounds

One aspect of the present disclosure provides a compound of Formula (I), a pharmaceutically acceptable salt, a hydrate, a prodrug, an ester, or a derivative thereof:

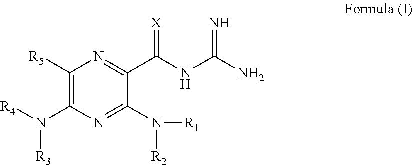

wherein R₁ and R₂ are independently H, C₁-C₈ alkyl, substituted C₁-C₈ alkyl, C₁-C₈ alkenyl, substituted C₁-C₈ alkenyl, C₃-C₆ substituted cycloalkyl, a C₃-C₆ unsubstituted cycloalkyl, aryl, or substituted aryl;
R₃ and R₄ are independently H, C₁-C₈ alkyl, substituted C₁-C₈ alkyl, C₁-C₈ alkenyl, substituted C₁-C₈ alkenyl, C₃-C₆ substituted cycloalkyl, a C₃-C₆ unsubstituted cycloalkyl, aryl, substituted aryl, or R³ and R⁴ may be taken together to form a C₃-C₆ substituted cycloalkyl, a C₃-C₆ unsubstituted cycloalkyl, C₃-C₆ substituted cycloalkenyl, a C₃-C₆ unsubstituted cycloalkenyl, a substituted heterocycle, a unsubstituted heterocycle, or a C₃-C₂₄ heteroaromatic;
R₅ is C₁-C₈ substituted alkyl, C₁-C₈ unsubstituted alkyl, C₃-C₆ substituted cycloalkyl, C₃-C₆ unsubstituted cycloalkyl, a substituted heterocycle, an unsubstituted heterocycle, C₃-C₂₄ aromatic, C₃-C₂₄ heteroaromatic,

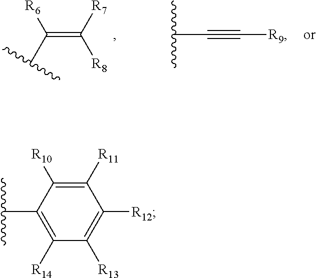

and
R₆, R₇, R₈, R₉, R₁₀, R₁₁, R₁₂, R₁₃, and R₁₄ independently are H, Cl, Br, F, CF₃, C₁-C₆ substituted alkyl, C₁-C₆ unsubstituted substituted alkyl, OCH₃, NH₂, NHCH₃, N(CH₃)₂, CN, cyclopropyl, cyclobutyl, cyclopentyl, cyclohexyl, or phenyl; and X is O, S, or NH.

In general, R₁ and R₂ may be independently H, C₁-C₄ alkyl, C₁-C₄ substituted alkyl, C₁-C₄ alkenyl, C₃-C₅ substituted cycloalkyl, a C₃-C₅ unsubstituted cycloalkyl, aryl, or substituted aryl. R₃ and R₄ may be independently H, C₁-C₄ alkyl, C₁-C₄ substituted alkyl, C₁-C₄ alkenyl, C₃-C₅ substituted cycloalkyl, a C₃-C₅ unsubstituted cycloalkyl, aryl, substituted aryl, or R³ and R⁴ may be taken together to form a C₃-C₅ substituted cycloalkyl, a C₃-C₅ unsubstituted cycloalkyl, C₄-C₁₄ heteroaromatic, a substituted heterocycle, or an unsubstituted heterocycle. In some embodiment, R₁, R₂, R₃, and R₄ may be independently H, CH₃, CH₃CH₂, CH(CH₃)₂, C(CH₃)₃, or phenyl. In certain embodiments, R₁, R₂, R₃, and R₄ may be independently H or CH₃. Particularly, R₁ and R₂ may be H; R₃, and R₄ may be CH₃.

In general, R₅ may be C₁-C₄ substituted alkyl, C₁-C₄ unsubstituted alkyl, C₃-C₅ substituted cycloalkyl, C₃-C₅ unsubstituted cycloalkyl, C₄-C₁₄ aromatic, C₄-C₁₄ heteroaromatic, a substituted heterocycle, an unsubstituted heterocycle,

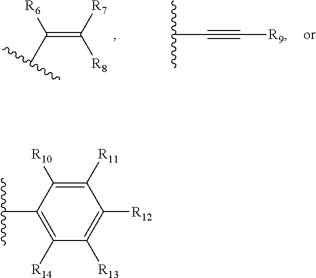

In certain embodiments, R₅ may be

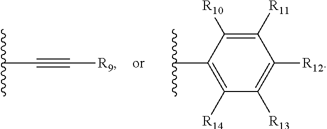

### Particularly,

R₅ may be

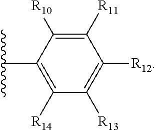

Still particularly, R₅ may be

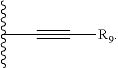

In general, R₆, R₇, R₈, R₉, R₁₀, R₁₁, R₁₂, R₁₃, and R₁₄ may be independently H, Cl, Br, F, OCH₃, NH₂, NHCH₃, N(CH₃)₂, or phenyl. In some embodiments, R₆, R₇, R₈, R₉, R₁₀, R₁₁, R₁₂, R₁₃, and R₁₄ may be independently H, CH₃, F, CF₃, or phenyl. In certain embodiments, R₉, R₁, R₁₁, R₁₂, R₁₃, and R₁₄ may independently be H or phenyl. Particularly, R₁₀, R₁₁, R₁₂, R₁₃, and R₁₄ may be H. Particularly, R₉ may be phenyl. Particularly, R₁₀, R₁₁, R₁₃, and R₁₄ may be H and R₁₂ may be phenyl.

Generally, X may be O.

In some embodiments, the compound may be DMA-132, wherein R₁ and R₂ are H; R₃, and R₄ are CH₃, R₅ is

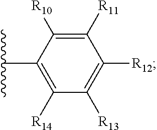

and R₁₀, R₁₁, R₁₂, R₁₃, and R₁₄ are H as shown in the compound of Formula (II):

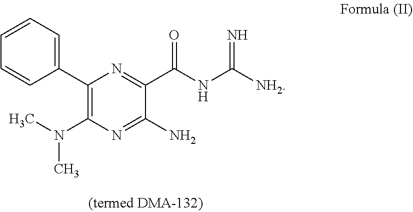 [SMILES: CN(C)c1nc(N)c(C(=O)NC(=N)N)nc1-c1ccccc1]

In some embodiments, the compound may be DMA-135, wherein R₁ and R₂ are H; R₃, and R₄ are CH₃, R₅ is

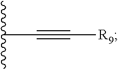

and R₉ is phenyl as shown in the compound of Formula (III):

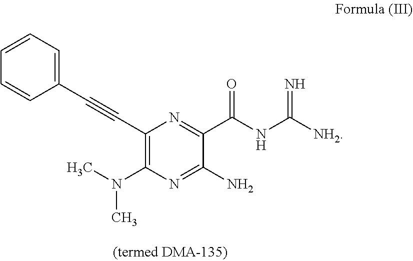 [SMILES: CN(C)c1nc(N)c(C(=O)NC(=N)N)nc1C#Cc1ccccc1]

In some embodiments, the compound may be DMA-155, wherein R₁ and R₂ are H; R₃, and R₄ are CH₃, R₅ is

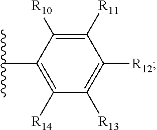

R₁₀, R₁₁, R₁₃, and R₁₄ are H; R₁₂ is phenyl as shown in the compound of Formula (IV):

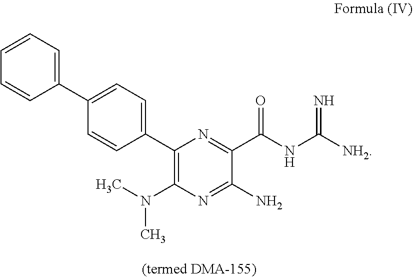 [SMILES: CN(C)c1nc(N)c(C(=O)NC(=N)N)nc1-c1ccc(-c2ccccc2)cc1]

B. Pharmaceutical Compositions

In another aspect, the present disclosure provides compositions comprising one or more of the compounds as described above and herein and an appropriate carrier, excipient or diluent. The exact nature of the carrier, excipient or diluent will depend upon the desired use for the composition, and may range from being suitable or acceptable for veterinary uses to being suitable or acceptable for human use. The composition may optionally include one or more additional compounds.

When used to treat or prevent a disease, such as a viral infection, the compounds described herein may be administered singly, as mixtures of one or more compounds or in mixture or combination with other agents (e.g., therapeutic agents) useful for treating such diseases and/or the symptoms associated with such diseases. Such agents may include, but are not limited to, antiviral agents, NSAIDS, and the like. The compounds may be administered in the form of compounds per se, or as pharmaceutical compositions comprising a compound.

Pharmaceutical compositions comprising the compound(s) may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the compounds into preparations which can be used pharmaceutically.

The compounds may be formulated in the pharmaceutical composition per se, or in the form of a hydrate, solvate, N-oxide or pharmaceutically acceptable salt, as previously described. Typically, such salts are more soluble in aqueous solutions than the corresponding free acids and bases, but salts having lower solubility than the corresponding free acids and bases may also be formed.

Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

For topical administration, the compound(s) may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the compound, as is well known. For buccal administration, the compositions may take the form of tablets or lozenges formulated in conventional manner. For rectal and vaginal routes of administration, the compound(s) may be formulated as solutions (for retention enemas) suppositories or ointments containing conventional suppository bases such as cocoa butter or other glycerides.

For nasal administration or administration by inhalation or insufflation, the compound(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For ocular administration, the compound(s) may be formulated as a solution, emulsion, suspension, etc. suitable for administration to the eye. A variety of vehicles suitable for administering compounds to the eye are known in the art.

For prolonged delivery, the compound(s) can be formulated as a depot preparation for administration by implantation or intramuscular injection. The compound(s) may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the compound(s) for percutaneous absorption may be used. To this end, permeation enhancers may be used to facilitate transdermal penetration of the compound(s).

Alternatively, other pharmaceutical delivery systems may be employed. Liposomes and emulsions are well-known examples of delivery vehicles that may be used to deliver compound(s). Certain organic solvents such as dimethyl sulfoxide (DMSO) may also be employed, although usually at the cost of greater toxicity.

The pharmaceutical compositions may, if desired, be presented in a pack or dispenser device which may contain one or more-unit dosage forms containing the compound(s). The pack may, for example, comprise metal or plastic foil, such as a blister pack. The pack or dispenser device may be accompanied by instructions for administration.

The compound(s) described herein, or compositions thereof, will generally be used in an amount effective to achieve the intended result, for example in an amount effective to treat or prevent the particular disease being treated. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

The amount of compound(s) administered will depend upon a variety of factors, including, for example, the particular indication being treated, the mode of administration, whether the desired benefit is prophylactic or therapeutic, the severity of the indication being treated and the age and weight of the patient, the bioavailability of the particular compound(s) the conversation rate and efficiency into active drug compound under the selected route of administration, etc.

Determination of an effective dosage of compound(s) for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound that is at or above an IC₅₀ of the particular compound as measured in as in vitro assay. Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages of particular compounds suitable for human administration.

Dosage amounts will typically be in the range of from about 0.0001 mg/kg/day, 0.001 mg/kg/day or 0.01 mg/kg/day to about 100 mg/kg/day, but may be higher or lower, depending upon, among other factors, the activity of the active compound, the bioavailability of the compound, its metabolism kinetics and other pharmacokinetic properties, the mode of administration and various other factors, discussed above. Dosage amount and interval may be adjusted individually to provide plasma levels of the compound(s) and/or active metabolite compound(s) which are sufficient to maintain therapeutic or prophylactic effect. For example, the compounds may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

### C. Methods and Uses

The compounds provided herein have many uses. Hence, one aspect of the present disclosure provides a method inhibiting viral replication in a cell and/or subject. This method may comprise administering to the cell and/or subject an effective amount of any of the compounds or the pharmaceutical composition described above and herein such that the viral replication is inhibited in the cell and/or subject

Another aspect of the present disclosure provides a method of preventing or treating a viral infection in a subject. This method may comprise administering to the subject an effective amount of any of the compounds or the pharmaceutical composition described above and herein such that the viral infection is prevented or treated in the subject.

In some embodiments, the virus may comprise a coronavirus. In some embodiments, the coronavirus may be selected from the group consisting of 229E (alpha coronavirus), NL63 (alpha coronavirus), OC43 (beta coronavirus), HKU1 (beta coronavirus), MERS-CoV (beta coronavirus), SARS-CoV (beta coronavirus), SARS-CoV-2, and combinations thereof. In certain embodiments, the coronavirus may comprise SARS-CoV-2.

Another aspect of the present disclosure provides a method of preventing or treating COVID-19 in a subject. This method may comprise administering to the subject an effective amount of any of the compounds or the pharmaceutical composition described above and herein such that COVID-19 is prevented or treated in the subject.

Still another aspect of the present disclosure provides use of any of the compounds or the pharmaceutical composition described above and herein as an antiviral agent. In some embodiments, any of the compounds or the pharmaceutical composition described above and herein may be used in conjunction with another antiviral or anti-inflammatory agent. In certain embodiments, the agents may be combined in the same formulation or pill.

In some embodiments, the antiviral agent targets coronavirus RNA. By way of non-limiting example, the coronavirus may comprise 229E (alpha coronavirus), NL63 (alpha coronavirus), OC43 (beta coronavirus), HKU1 (beta coronavirus), MERS-CoV (beta coronavirus), SARS-CoV (beta coronavirus), SARS-CoV-2, or a combination thereof. In certain embodiments, the coronavirus may comprise SARS-CoV-2.

In some embodiments, for any of the methods described above and herein, the compound or the pharmaceutical composition may be administered in conjunction with another antiviral or anti-inflammatory agent. In certain embodiments, the compound or the pharmaceutical composition and another antiviral or anti-inflammatory agent may be combined in the same formulation or pill.

Yet another aspect of the present disclosure provides use of any of the compounds or the pharmaceutical composition described above and herein as an antiviral agent for preventing or treating COVID-19. In some embodiments, any of the compounds or the pharmaceutical composition described above and herein may be used in conjunction with another antiviral or anti-inflammatory agent. In certain embodiments, the agents may be combined in the same formulation or pill.

The following Examples are provided by way of illustration and not by way of limitation.

### A. Materials and Methods

Analysis of Sequence Conservation: Representative betacoronavirus sequences were selected according to official taxonomy as represented by the International Committee for the Taxonomy of Viruses (Lefkowitz et al., Nucleic acids research 46, D708-D717 (2018)). Multiple sequence alignments of coronavirus sequences were constructed using BLAST and MAAFT as implemented in GLUE (Altschul et al., Nucleic acids research 25, 3389-3402 (1997); Katoh and Standley, Mol Biol Evol 30, 772-780 (2013); Singer et al., BMC Bioinformatics 19, 532-532 (2018)). Alignments were manually inspected and adjusted using Se-Al. Position coverage and percentage identity were calculated and visualized using JalView (Waterhouse et al., Bioinformatics (Oxford, England) 25, 1189-1191 (2009)).

Cells and Virus: Vero E6 (African green monkey kidney; ATCC CRL-1586) cells were cultured in MEM medium supplemented with 10% FBS (ThermoFisher Scientific) and maintained at 33° C. Cells were infected with human coronavirus OC43 (ATCC VR-1558) at indicated MOI (multiplicity of infection) and incubated 1 hr at 33° C. for adsorption. Unbound virus was removed, and cells were refed fresh medium with various concentrations of DMAs. Media from infected cells were harvested 24 hrs post-infection, and virus titers were determined by plaque assay on Vero E6 cells. SARS-CoV-2 (USA-WA1/2020 strain; BEI Resources) was propagated and titered on VeroE6 cells, with sequence confirmation of a P2 stock to confirm stability of the viral genome.

Effects of SARS-CoV-2 5′- and 3′-End Sequence Elements on Luciferase Reporter Activity: The reporter plasmid pCoV-2-5′UTR-FLuc-3′UTR contains the SARS-CoV-2 5′UTR and adjacent coding sequences in ORFla fused in-frame with the firefly luciferase open reading frame, followed by the SARS-CoV-2 3′UTR. For reporter plasmid pCoV-2-5′UTR-FLuc, the Cov-2 3′UTR as replaced with vector-encoded sequence. The plasmids were kindly provided by Dr. Shin-Ru Shih (Chang-Gung University, Taiwan). Plasmid pRL, the Renilla luciferase control reporter vector, was purchased from Promega. CoV-2-5′UTR-FLuc-3′UTR, Cov-2-5′UTR-FLuc, and RLuc RNAs were in vitro synthesized from these plasmid templates using the MEGAscript T7 Transcription Kit (ThermoFisher Scientific).

Vero E6 cells were seeded in 24-well plates. Two hundred (200) ng of reporter RNA, 5 μl of SuperFect (Qiagen), and 400 μl of MEM with 10% FBS were combined and added to one well of cells. Cells were incubated at 33° C. for 4 hrs and media were changed, and various concentrations of DMAs were added. Two days after transfection, reporter luciferase activities were determined by measuring Renilla luciferase (RLuc) and firefly luciferase (FLuc) activities using the Dual Luciferase Reporter Assay System (Promega).

Cytotoxicity Assays: Various concentrations of DMAs were added to Vero E6 cells in culture. The cells were incubated at 33° C. for 96 hrs. Cell viability was determined by MTT assay and measured at 570 nm according to the manufacturer's instructions (EMD Millipore). All experiments were performed in triplicate. The concentration of DMAs required to reduce cell viability to 50% of the control cells was expressed as CC₅₀.

SARS-CoV-2 Antiviral Assays: Analysis of SARS-CoV-2 growth in cell culture supernatants was performed using a simplified RT-qPCR assay as explained before (Mugisha et al., mSphere 5, e00658-20 (2020)). In brief, Vero E6 cells were infected with SARS-CoV-2 at an MOI of 0.1 i.u./cell in 96-well plates, virus inoculum was removed 1 hour post-adsorption and replaced with media containing serial dilutions of the DMA compounds. 5 μL of cell culture media containing released virions were collected at 24 hpi and processed as detailed in previous studies. Viral RNA levels were quantitated by RT-qPCR using primers specific to SARS-CoV-2 N gene and a standard curve derived from in vitro synthesized RNA encoding N.

In addition, antiviral activity was tested against SARS-CoV-2 using a TCID₅₀ assay, VeroE6 cells were seeded at 1×10⁵ per well in a 24-well plate at 37° C. for 24 hours. Cells and samples were then transferred to a Biosafety Level 3 facility. Stocks of SARS-CoV-2 were diluted in DMEM/2% FBS for a solution of 20,000 pfu/mL. Growth media was aspirated from 24-well plates and replaced with 495 μL of DMEM/2% FBS containing 20,000 pfu/mL SARS-CoV-2 for an M.O.I. of 0.1. 5 μL of DMSO or compound were then immediately diluted into each well for final concentrations of 50 μM and 10 μM. Plates were incubated at 37° C. for 72 hours. Media was then harvested, centrifuged at RT for 10 min at 1,500×g, then used for TCID50 assay. Serial dilutions of supernatant from the treated cells were added to VeroE6 cells in 96 well plates and cells are monitored for cytopathic effect (CPE). Viral titer was calculated from the numbers of positive wells using a modified Reed and Muench method.

Synthesis and Purification of RNA Stem Loop Constructs Present at 5′-UTR: SL1 through 6 of the 5′-end were in vitro transcribed using a standard protocol, from synthetic DNA templates from Integrated DNA Technologies (Coralville, IA) (Milligan and Uhlenbeck, Methods Enzymol 180, 51-62 (1989); Milligan et al., Nucleic Acids Research 15, 8783-8798 (1987)). The 3-6 ml reactions involved the use of purified recombinant T7 RNA polymerase expressed in BL21 (DE3) cells. Depending on the labeling scheme of each RNA, double labeled, ¹³C and ¹⁵N, rNTPs and unlabeled rNTPs were utilized in the reaction. Nucleotide labeling of the SLs was based on the abundance of nucleotides in bulges and loops. The labeling pattern for the ¹H-¹³C HSQC experiments was as follows: SL1 C(¹³C, ¹⁵N)-labeled and A(¹³C, ¹⁵N)-labeled, SL2 U(¹³C, ¹⁵N)-labeled, SL3 AU(¹³C, ¹⁵N)-labeled, SL4 A(¹³C,¹⁵N)-labeled, SL5a AU(¹³C, ¹⁵N)-labeled, SL5b AU(¹³C, ¹⁵N)-labeled, and SL6 AC(¹³C, ¹⁵N)-labeled. For the ¹H-¹H NOESY experiments, the labeling scheme was SL6 UG(²H) AC(²H_{3′-5″})-, CUG(²H) A(²H_{3′-5″})-, and ACUG(²H)-selectively labeled constructs, along with a CG(²H), AU(²H_{3′-5″}-)-selectively labeled SL1 construct.

Next, the SLs were purified on denaturing gels, ranging from 8 to 16%, and extracted using electroelution. After desalting with a Millipore Amicon Ultra-4 centrifugal filter, the RNAs were annealed by heating for 2 min at 95° C. and flash-cooled on ice. The samples were thoroughly washed and concentrated down with a 100% D₂O buffer of 25 mM K₂HPO₄, 50 mM KCl at pH 6.2. Using the NanoDrop™ 2000 software (Thermo Fisher), the theoretical extinction coefficients of the SLs were calculated in order to determine RNA concentrations. Samples for NMR titrations contained 100 μM of RNA in D₂O buffer with a final volume of 200 μL.

NMR Profiling of DMA Interactions with 5′-End Structures: A 900 MHz spectrometer was used to record all NMR data. The ¹H-¹³C HSQC titrations were recorded with 100 μM of selectively labeled SL1 through 6 in a 100% D₂O buffer of 25 mM K₂HPO₄, 50mM KCl at pH 6.2 with a 200 μL sample volume. Titrations of the DMA molecules into the different SLs were collected at a molar ratio of 5:1, DMA to RNA, at a temperature of either 298, 303, or 308K. For each construct, temperature optimization experiments were done in order to determine the optimum temperature to conduct the titrations with. Also, based on the optimized temperatures, ¹H-¹H NOESY spectra (tₘ=250 ms) of selectively labeled SL6 samples and their DMA-155 complexes were collected with a 200 μM sample concentration in 25 mM K₂HPO4, 50 mM KCl pH 6.2 in 100% D2O at a 200 μL sample volume. At similar conditions, ¹H-¹H NOESY spectra (tₘ=250 ms) of selectively labeled SL1 were collected in the absence and presence of DMA-135 and 155 at a 5:1 molar ratio. The NMR spectra were processed using NMRPipe/NMRDraw and analyzed with NMRView J or Sparky (Delaglio et al., Journal of Biomolecular NMR 6, 277-293 (1995); Johnson and Blevins, Journal of Biomolecular NMR 4, 603-614 (1994); Lee et al., Bioinformatics 31, 1325-1327 (2014)).

Virtual Ligand Screening against SARS-CoV-2 5′-End RNA Structures:

FARFAR model generation: FARFAR is part of the Rosetta 3 software package can be obtained for free academic usage (https://www.rosettacommons.org/software/license-and-download). Each SL was generated through the following protocol.

rna_helix.py is a python wrapper for the Rosetta executable rna_helix. rna_helix.py is available in $ROSETTA/tools/rna_tools/bin, where $ROSETTA is the Rosetta installation path. Below are the commands to generate SL1.

rna_helix.py-seq acc ggu-resnum 8-10 17-19-o helix_2.pdb

For each helix in a SL other than the nucleotides that flank bulges and loops we prebuild as idealized A-form helices with the above commands. Modeling helical residues as idealized A-form significantly reduces computational time allowing for more models to be built.

FARFAR modeling is performed through the rna denovo executable.

rna_denovo -nstruct 1000 -s helix_1.pdb helix_2.pdb -fasta input.fasta-secstruct_file input.secstruct -minimize_rna true -out.file: silent farfarout

where -nstruct is the max number of models requested, -fasta is the path of a fasta file containing the RNA sequence, -secstruct is the path of a file (input. secstruct) containing the RNA secondary structure in dot-bracket notation, -minimize rna true minimizes the RNA after fragment assembly, -s specifies the path to the pdb files that contain static structures of our helices, and -out:file:silent specifies the output file path to store all generated models. Each SL was run on 100 cores for 24 hours, the number of generated models is reported in table FARFAR (Table 1).

TABLE 1
Binding pockets identified with ICM pocket finder and characterization of each binding pocket.

| SL | Volume | Area | Hydrophobicity | Buriedness | Aromatic | DLID | Radius | Nonsphericity |
|---|---|---|---|---|---|---|---|---|
| 1.4 | 132.87 | 143.87 | 0.47 | 0.86 | 0.05 | −0.63 | 3.17 | 1.14 |
| 1.2 | 111.15 | 211.04 | 0.26 | 0.56 | 0.00 | −2.42 | 2.98 | 1.89 |
| 1.1 | 108.75 | 234.70 | 0.20 | 0.56 | 0.04 | −2.59 | 2.96 | 2.13 |
| 3 | 111.58 | 124.56 | 0.52 | 0.85 | 0.00 | −0.72 | 2.99 | 1.11 |
| 4.1 | 583.35 | 615.91 | 0.37 | 0.80 | 0.09 | −0.02 | 5.18 | 1.82 |
| 4.2 | 142.86 | 192.83 | 0.35 | 0.68 | 0.02 | −1.55 | 3.24 | 1.46 |
| 4.3 | 126.79 | 168.48 | 0.40 | 0.78 | 0.08 | −1.15 | 3.12 | 1.38 |
| 5a.1 | 273.58 | 474.91 | 0.37 | 0.74 | 0.03 | −0.81 | 4.03 | 2.33 |
| 5a.2 | 174.42 | 274.74 | 0.37 | 0.70 | 0.01 | −1.29 | 3.47 | 1.82 |
| 5a.3 | 160.57 | 228.90 | 0.38 | 0.69 | 0.01 | −1.37 | 3.37 | 1.60 |
| 5a.4 | 133.51 | 206.26 | 0.31 | 0.64 | 0.00 | −1.86 | 3.17 | 1.63 |
| 5a.5 | 132.20 | 180.61 | 0.42 | 0.70 | 0.06 | −1.39 | 3.16 | 1.44 |
| 5b | 285.30 | 310.08 | 0.49 | 0.85 | 0.14 | −0.04 | 4.08 | 1.48 |
| 6.2 | 235.09 | 215.04 | 0.61 | 0.95 | 0.11 | 0.45 | 3.83 | 1.17 |
| 6.1 | 219.49 | 239.54 | 0.57 | 0.76 | 0.20 | −0.41 | 3.74 | 1.36 |

To reduce the number of models to dock against, fixed-width clustering using the rna_cluster executable was performed.

rna_cluster -in:file:silent farfar.out -nstruct 15 -cluster:radius RADIUS

Where -in:file:silent is a silent file of all models for a given SL, -nstruct is the max number of clusters requested, and -cluster:radius is max distance in heavy-atom RMSD between members of the same cluster.

Small Molecule Docking: Virtual docking simulations were performed using ICM (Abagyan et al., Journal of Computational Chemistry 15, 488-506 (1994)) (Molsoft LLC. La Jolla, CA), employing the SARS-CoV-2 5′-UTR structures obtained from FARFAR modeling and clustering. RNA structural elements' binding pockets were defined using ICM Pocket Finder module and all the small molecules protonation states were adjusted to pH=7.0 using ChemAxon© (www.chemaxon.com). RNA ensembles were then combined into conformational stacks using ICM's “impose conformations.” Then, to create “flexible receptors” to dock against that would reflect all of the conformations of each structure, ICM's “create 4D grid” function was used for each docking project. Each of the structures was then docked against a library of 55 DMA molecules. The DMA library was saved in .sdf file format, which was indexed for VLS using ICM-Pro. The virtual screening simulation was implemented with a conformational search and optimization with a limit of 10 conformers per molecule. The thoroughness was left at level 10.

Indicator Displacement Assay (IDA): A serial dilution of the seven SLs RNA (SL1, SL2, SL3, SL4, SL5a, SL5b, and SL6) was performed in Tris buffer (50 mM Tris-HCl, 50 mM KCl, at pH 7.4) in a 96 well plate in triplicate. 8 μL of each dilution were transferred to a 384 well plate followed by 8 uL of a 500 nM solution of RiboGreen™ dye (Invitrogen) in the same buffer. The plates were excited at 487 nm (8 nm slit) and emission was recorded at 525 nm (8 nm slit, focal height 11.3 mm) using a CLARIOstar plate reader (BMG labtech). The affinity of the dye for the RNA construct was determined by fitting the raw fluorescence in in GraphPad Prism version 8.3.1 for Macintosh (GraphPad Software, La Jolla California USA, (www.graphpad.com)) by fitting to the [Agonist] vs. response—variable slope model that using Equation (1).

Y=Bottom+(X\hat{ }HillSlope)*(Top—Bottom)/(X\hat{ }HillSlope+EC50\hat{ }HillSlope)−Equation (1)

Where Y is normalized % change in fluorescence intensity, X is RNA concentration, Bottom is lowest fluorescence % change and Top is highest fluorescence % change. Affinity of the dye for the RNA construct was used as the ideal RNA concentration for small molecule titrations.

The two most promising small molecule leads (DMA-135 and -155) were then screened against SL1, SL4, SL5a, SL5b, and SL6 but not against the short stem loops SL2 and SL3 for which the RiboGreen™ dye showed weak affinity.

A serial dilution of DMA-135 and -155 (0, 0.5, 1.0, 2.5, 5, 10.0, 12.5, 15, 20, 25, 40, 50, 75, 100, 225, 400, 450 μM) was performed in phosphate buffer Tris buffer (50 mM Tris-HCl, 50 mM KCl, at pH 7.4) in a 96 well plate in triplicate. 8 μL of each dilution were transferred to a 384 well plate, followed by 8 μL of a solution of Stem loop RNA and 0.5 μM of RiboGreen™ (Invitrogen). The 384 well plates were at 4000 rpm for 1 min, and incubated in the dark for 30 min. The plates were excited at 487 nm (8 nm slit) and emission was read at 525 nm (8 nm slit, focal height 11.3 nm) using a CLARIOstar plate reader (BMG Labtech). Percent fluorescence indicator displacement (%FID) was calculated by subtracting and, subsequently, dividing by the blank wells with RNA-dye complex and no small molecule as shown in Equation (2).

$$\% FID = ( \frac{{F}_{0} - F}{{F}_{0}} ) * 100 \qquad Equation ( 2 )$$

Where F₀ is the fluorescence of the blank well with RNA+dye and no small molecule and F is the fluorescence of the well with all three components (RNA+dye+small molecule).

Each technical triplicate was averaged, and the resulting FID values were averaged between three independent experiments. The binding curve and EC₅₀ value was obtained by using a non-linear fit curve agonist vs. response with variable slope at four parameters (GraphPad Prism Software version for Macintosh 8.3.1, La Jolla, California, USA (www.graphpadprism.com)) as shown in Equation (1). Reported values are averages of three independent experiments ±standard deviation.

### B. Introduction

Current candidates for treatment have limited approval for emergency use in severe COVID-19 cases. Remdesivir, for example, is an RNA-dependent RNA polymerase inhibitor (RdRp) initially developed during the Ebola outbreak and revisited at the start of the pandemic (Shannon et al., Antiviral Res 178, 104793-104793 (2020)). The compassionate use of the candidate antiviral across many countries reported mixed results, with overall faster recovery time from the virus but no difference in mortality rates (Singh et al., Diabetes Metab Syndr 14, 641-648 (2020)). While more randomized trials are needed for a final verdict on the efficacy of Remdesivir in critical patients, its stereospecific multi-step synthetic process highlights the need for new, scalable and more efficacious antivirals. Baricitinib has been recently approved for emergency use for COVID-19 treatment in conjunction with Remdesivir. Also known as Olumiant, this small molecule was approved in 2018 as treatment for moderate to severe rheumatoid arthritis (Genovese et al., The Lancet Rheumatology 2, e347-e357 (2020)). It is proposed that the anti-inflammatory effects of the drug help in decreasing inflammatory cascades associated with COVID-19. While promising, Baricitinib has yet to receive approval as a stand-alone treatment and, so far, has been shown to improve recovery time by one day when compared to Remdesivir alone treatment (Release, FDA news (2020)).

The recent emergence of multiple coronaviruses pandemics clearly indicates that SARS-CoV-2 most likely will not be the last CoV pandemic (Costa et al., Arch Virol 165, 1517-1526 (2020)). The current limited tools and lack of cures underscore the need for a new approach in developing antivirals that would not only provide novel routes to combat the current pandemic but also provide invaluable information on targetable structures that can aid in the prevention of and fight against future CoV outbreaks. Indeed, small molecules are uniquely poised to achieve this goal as their design and development provides both a better understanding of CoV biology and identify druggable targets that could aid in the development of pan-coronavirus antivirals.

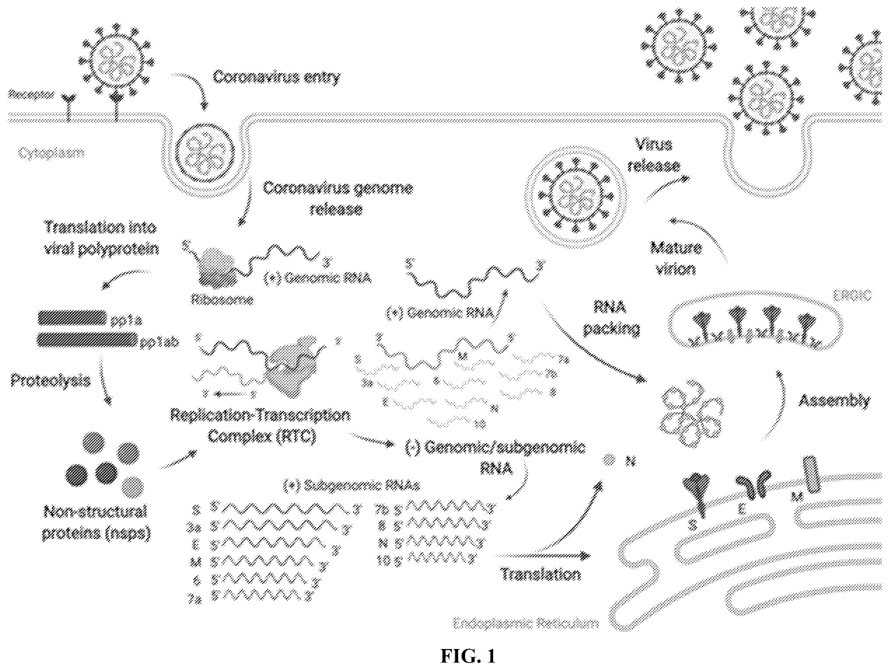

Several steps in the coronavirus replication cycle offer potential therapeutic targets for viral inhibition (FIG. 1). Coronaviruses are enveloped positive sense single-stranded RNA genomes of approximately 30 kilobases, making them the largest genomes of RNA viruses (Costa et al., Arch Virol 165, 1517-1526 (2020)). SARS-CoV-2 infects human cells by engagement of the ACE2 receptor by the viral spike (S) protein followed by membrane fusion at the plasma membrane or endosomal membranes depending on the availability of host cell proteases that cleave and prime S for entry. Fusion results in the release of its genome and associated proteins in the host cell cytosol. The genome is translated into two large polyproteins, 1a and 1b, which are then processed into individual proteins by the viral protease. Synthesis of full-length negative strand RNA by products of 1a/1b creates a template to synthesize multiple positive strand copies encapsidated by the viral nucleoprotein into virions (Costa et al., Arch Virol 165, 1517-1526 (2020)). The negative RNA strand also serves as a template for the synthesis of shorter sub-genomic RNAs (sgRNAs) that include the essential structural proteins and thus also constitute an attractive therapeutic target (Wu et al., Nature 579, 265-269 (2020)).

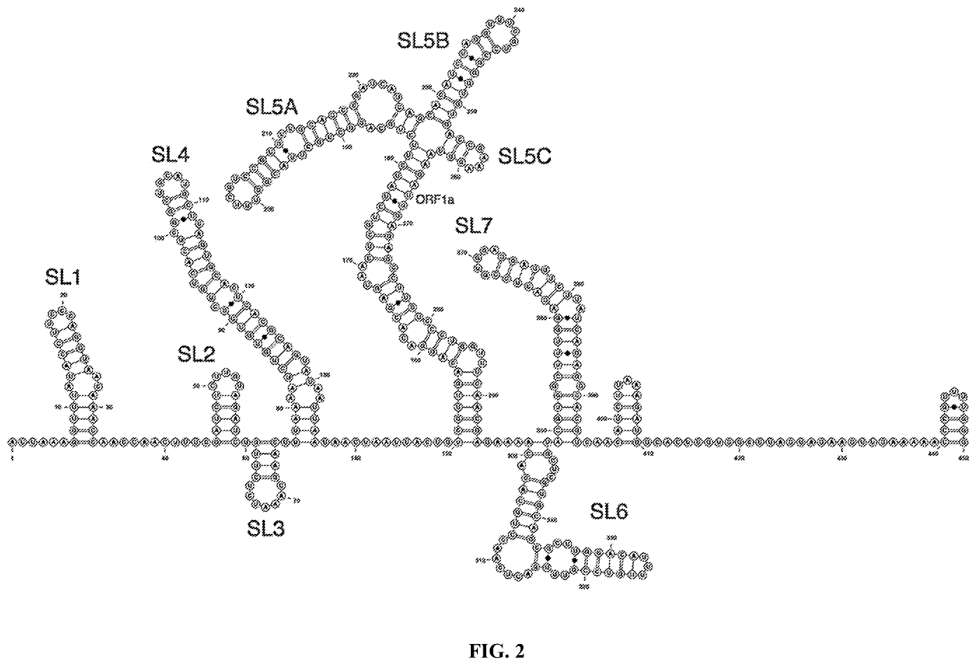

CoV antivirals to date have been developed to target viral proteins, including to prevent endocytosis, assembly of viral protein for export, and condensation of viral genome for packaging (Tiwari et al., Drug Discov Today 25, 1535-1544 (2020)). While this protein-centric approach has proven successful in a few cases, the sequence and structural conservation of RNA structural motifs pose an attractive complementary target for small molecule antiviral development, a strategy that has shown promise against a plethora of viruses (Nagy and Pogany, Nature Reviews Microbiology 10, 137-149 (2012); Hermann, Wiley Interdiscip Rev RNA 7, 726-743 (2016)). Specifically, international efforts that allowed for identification and tracking of SARS-CoV-2 variants highlighted the large number of mutations accumulated in protein-coding regions (Fang et al., Nucleic Acids Research 49, D706-D714 (2020)). At the same time, recent global cataloguing of mutations in the untranslated regions (UTRs) revealed a significantly lower rate of mutation, highlighting the potential of UTRs as drug targets (Mukherjee and Goswami, PLOS ONE 15, e0237559 (2020)). Recently published data on genome-wide secondary structure of the virus obtained by in vitro and in vivo SHAPE (selective 2′ hydroxyl acylation analyzed by primer extension) and DMS (dimethyl sulfate) probing of SARS-CoV-2 infected Vero E6 cells, as well as NMR characterization, recapitulates the computationally predicted stem loops (SL) at the 5′ region of the genome (FIG. 2) as well other relevant frameshifting and replication-related structures (Lan et al., bioRyiv, 2020.2006.2029.178343 (2020); Wacker et al., Nucleic Acids Research, 48, 12415-12435 (2020); Manfredonia et al., Nucleic Acids Research 48, 12436-12452 (2020); Huston et al., Molecular Cell 81, 584-598.e585 (2021)). Conserved elements at the 5′- and 3′-ends have been identified across many members of the coronavirus family and function as cis-acting elements regulating viral replication (Alhatlani, Future Virology 15, 409-417 (2020)). Specifically, studies on murine and bovine coronaviruses showed that phylogenetically conserved stem loops in the 5′-UTR are capable of long-range RNA-RNA and protein-RNA interactions responsible for optimal viral replication (Guan, et al., Journal of Virology 86, 4631-4643 (2012); Sola et al., RNA Biol 8, 237-248 (2011)). More recently, studies aimed at uncovering the pathway that leads to viral protein synthesis via host cell translation machinery revealed that the presence of the full length 5′-UTR of SARS-CoV-2 leads to a five-fold increase in translation of viral proteins. This preliminary data corroborates the importance of the 5′-UTR region, and the structures within, for efficient viral translation and provides context for the viral hijacking of the host cell translational machinery (Schubert et al., Nature Structural & Molecular Biology, 27, 959-966 (2020)).

Drug-like small molecules offer the ability to develop chemical probes that reveal function and to design bioavailable clinical candidates for treatment. While RNA targeting has lagged behind protein targeting, recent successes in both the laboratory and the clinic support its potential role. The first US FDA-approved small molecule targeting RNA other than the ribosome was approved for treatment of spinal muscular atrophy in August of 2020 (O'Keefe. (FDA Press Announcements 2020). Effective small molecule targeting in the laboratory has also been observed for a plethora of disease relevant RNAs, including viral RNAs. Specifically, small molecules targeting structures within the 5′-UTR region have shown antiviral activity for a number of positive-sense RNA viruses such as HCV, FMDV, and EV71 (Hermann, in RNA Therapeutics, A. L. Garner, Ed. (Springer International Publishing, Cham, 2018), pp. 111-134; Davila-Calderon et al., Nature Communications 11, 4775 (2020); Lozano et al., RNA Biol 12, 555-568 (2015)). Recent studies have begun evaluating the potential of small molecules against the frameshifting elements of SARS-CoV-2 RNA in the coding region. These recent reports include evaluation of known SARS-CoV pseudoknot binders as well as development of a small molecule binder to the attenuator hairpin preceding the pseudoknot (Kelly et al., J Biol Chem 295, 10741-10748 (2020); Bhatt et al., Science 372, 1306-1313 (2021); Sreeramulu et al., Angew Chem Int Ed Engl, 60, 19191-19200 (2021); Haniff et al., ACS Central Science 6, 1713-1721 (2020)). Coupling of the latter to the known ribonuclease targeting chimera (RIBOTAC) technology results in recruitment of cellular ribonucleases leading to viral genomic RNA degradation. In addition, a fragment-based screen against structures in the 5′-UTR of SARS-CoV-2 was successful in identifying potential ligands (Sreeramulu et al., Angew Chem Int Ed Engl, 60, 19191-19200 (2021)). While the antiviral activity of these molecules has not been published, these preliminary results, combined with known RNA-targeted antivirals for other positive sense RNA viruses, stand as proof-of concept of the targetability of SARS-CoV-2 RNA motifs.

Successful efforts to target viral 5′-UTR structures have often leveraged the synthetic tuning of a known RNA binding scaffold (Hermann, in RNA Therapeutics, A. L. Garner, Ed. (Springer International Publishing, Cham, 2018), pp. 111-134; Davila-Calderon et al., Nature Communications 11, 4775 (2020); Lefebvre et al., Biochemical and Biophysical Research Communications 455, 378-381 (2014)). This approach was employed to develop RNA-targeted antivirals based on dimethylamiloride (DMA) that target the internal ribosomal entry site (IRES) region in the 5′-UTR of EV71. The DMA scaffold had been previously reported to be poised for tuning for specific RNA constructs via a facile three-step synthesis (Davila-Calderon et al., Nature Communications 11, 4775 (2020)). Further investigation and functionalization of the scaffold resulted in a bioactive antiviral analogue that formed a repressive ternary complex with IRES stem loop II RNA and the human AUF1 protein, ultimately inhibiting translation and compromising viral replication. The successes in DMA exploration highlight the scaffold's potential for tuning as well as broad applicability as an antiviral scaffold.

The present disclosure reports DMA analogues that show promising antiviral properties by reducing SARS-CoV-2 viral titer in a dose-dependent manner in infected cells. In addition, dual luciferase reporter assays confirmed the antiviral activity of the small molecules to be dependent on the 5′-UTR and proximal region of the SARS-CoV-2 genome. Investigation of possible conserved RNA binding sites of the lead small molecules revealed putative bulge-like binding sites in stem loops 1, 4 and 5a, located in the 5′-UTR of the SARS-CoV-2 genome, as well as in the adjacent stem loop 6 located in ORFla, supporting both the targetability of 5′-region stem loops. Taken together, these results establish conserved CoV RNA structures as antiviral targets and reveal lead molecules with promising antiviral properties.

### Example 1

### Phylogenetical Conservation of RNA Structural Elements

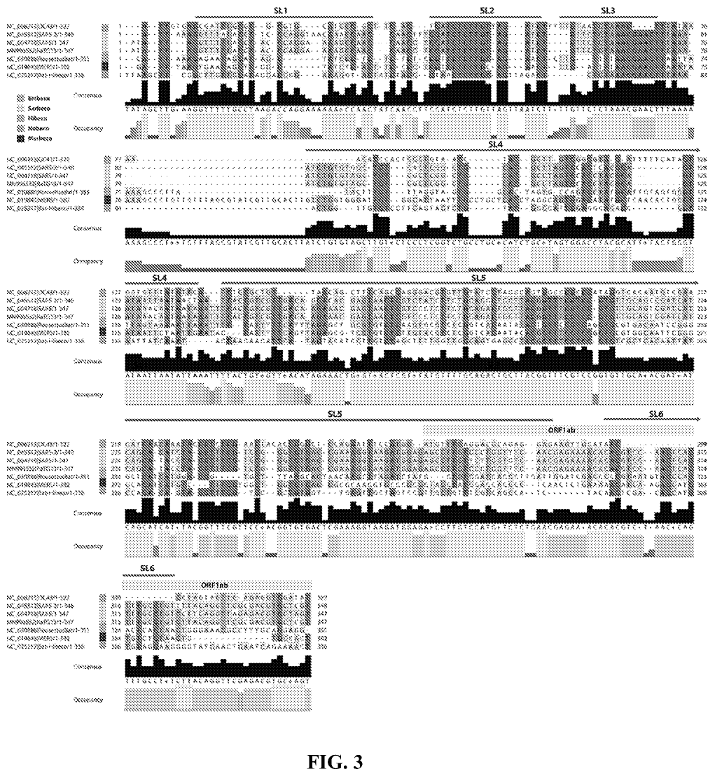

As the functional significance of the 5′-end stem loops (SLs) is still being elucidated, sequence conservation was examined in the 5′-end region across the Betacoronavirus genus as a preliminary approach to assessing the suitability of the known structures in this region as therapeutic targets. Sequence conservation within the contexts of folded RNA domains would imply selective pressures to exert biological function. A multiple sequence alignment spanning the 5′-UTR and the adjacent region including representatives of all five betacoronavirus subgenera was constructed (FIG. 3). Alignments disclosed the highest degree of conservation in the region encoding SLs 2-3, both of which are relatively short and contain stretches of 5-6 nucleotides that are 100% conserved. By contrast, SL1 and SLs 4-6 are less conserved, but notably, four of the five SLs that span the 5′-UTR contain stretches of relatively highly conserved nucleotide sequence (i.e. 70-100% conservation across the genus). The position of these SL's within or adjacent to the 5′-UTR (which is relatively conserved in length across all betacoronaviruses) indicates these conserved regions are likely to represent homologous nucleotides and suggests they are to some degree functionally equivalent. Our findings are in agreement with recent studies that reported sequence and structural similarity amongst members of the Coronaviridae family, suggesting that selective pressure plays a central role in conserving RNA secondary structures essential for the viral lifecycle (Davila-Calderon et al., Nature Communications 11, 4775 (2020)).

### Example 2

### Inhibition of Human Coronavirus OC43 Virus Replication by DMAs

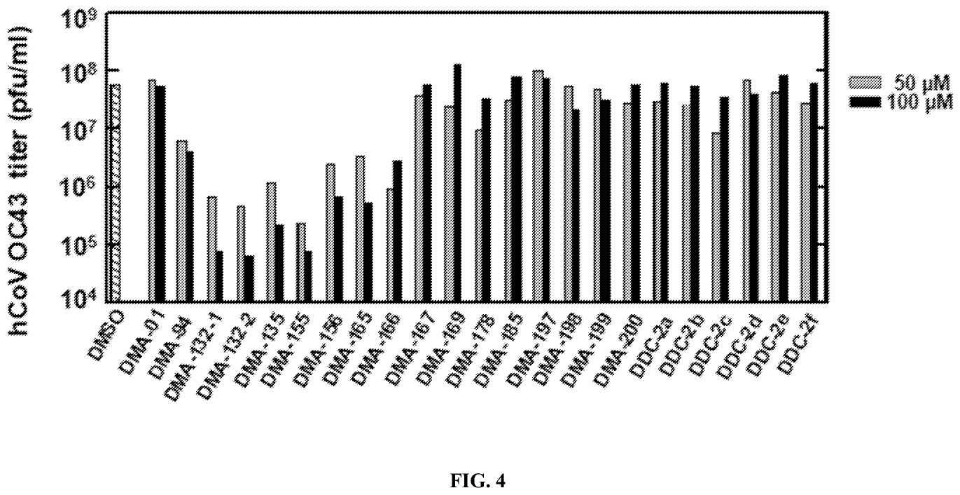

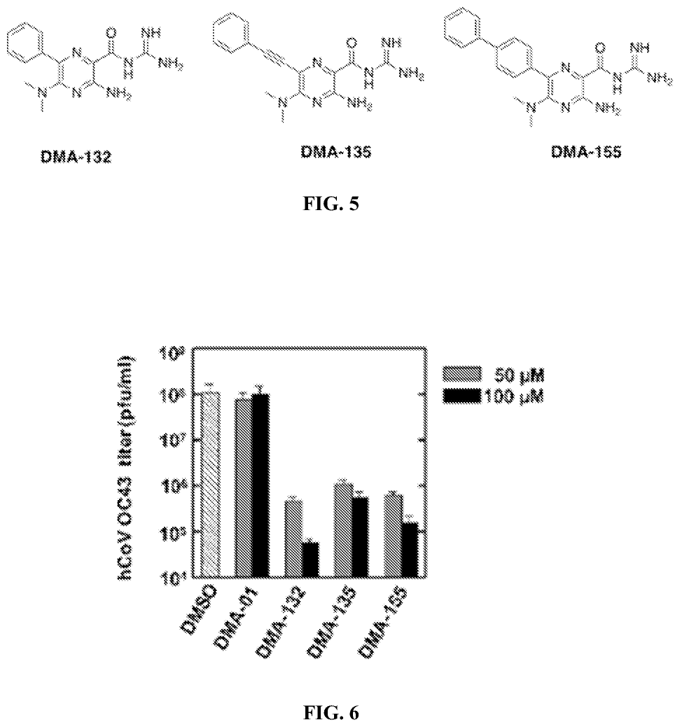

Targeting of the 5′-end stem loop structures with the DMA scaffold was pursued, which has been previously reported as an RNA binding scaffold that can be successfully optimized for selectivity for distinct RNA elements (Davila-Calderon et al. , Nature Communications 11, 4775 (2020)). To quickly assess potential CoV antiviral activity, human OC43 betacoronavirus was used due to its lower virulence and thus suitability for use in standard cell culture facilities (Chen et al., Signal Transduction and Targeted Therapy 5, 89 (2020)). Vero E6 cells were infected with human coronavirus OC43 at an MOI=1. After a one-hour adsorption, a panel of twenty-three DMAs at 50 μM or 100 μM were added to the cells and incubated for 24 hours at 33° C. Virus titers were determined by plaque formation on Vero E6 cells. The results of small molecule screening against OC43 with Vero E6 cells are shown in FIG. 4. Among the molecules screened, the chemical structures of the three lead molecules DMA-132, DMA-135, and DMA-155 are shown in FIG. 5. These molecules reduced virus titer by ˜1,000-fold at 100 μM concentration (FIG. 6). The results further suggest DMA-132, DMA-135, and DMA-155 reduce virus titer in a dose-dependent manner. The parent scaffold, dimethylamiloride (DMA-1), demonstrated no activity and is used as an inactive control moving forward.

### Example 3

### Antiviral Potency of DMAs against SARS-CoV-2

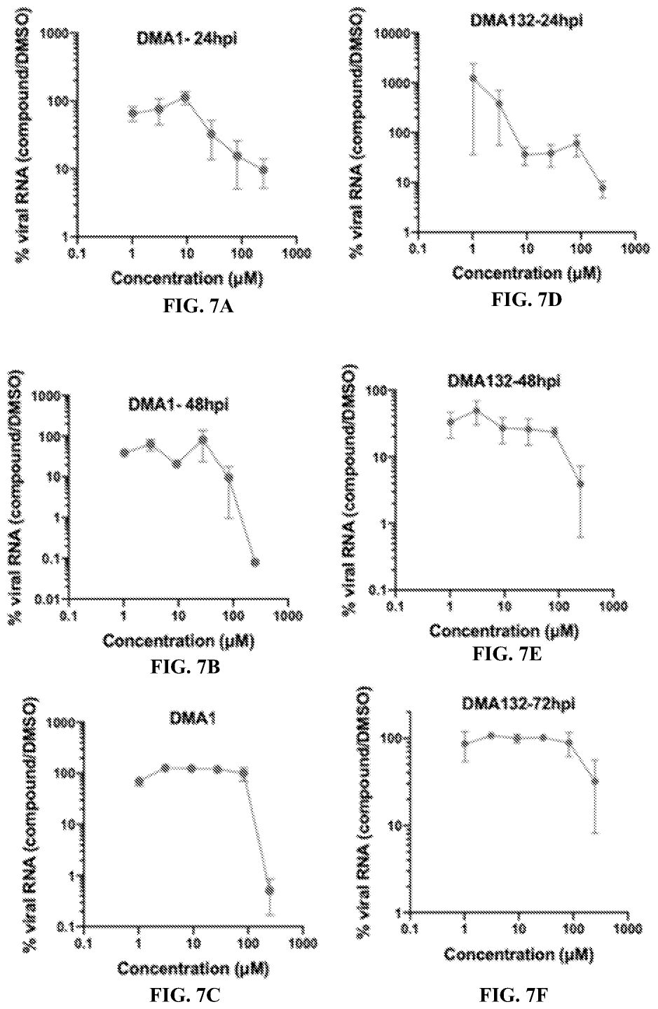

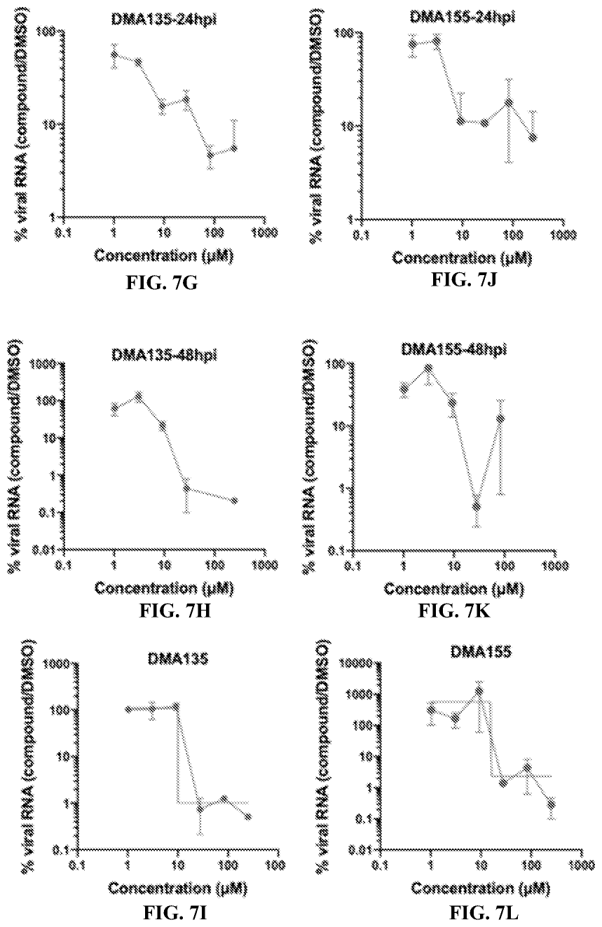

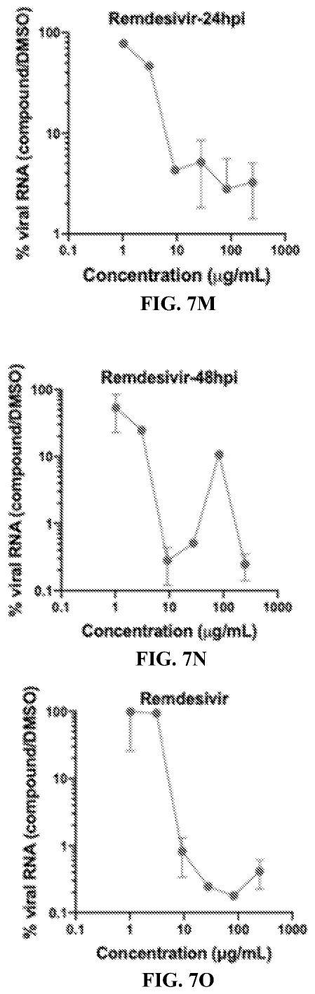

To determine the antiviral activity and potency of the lead small molecules against SARS-CoV-2, a simplified qRT-PCR assay was utilized to monitor SARS-CoV-2 viral RNA levels in supernatants of infected Vero E6 cells (Mugisha et al., mSphere 5, e00658-20 (2020)). Similar to Remdesivir, DMA-135 and DMA-155 led to a dose dependent 10-30-fold decrease in cell-free viral RNA levels within 24 hours of infection with an approximate IC₅₀ of 10 and 16 μM, respectively (FIGS. 7A-7O).

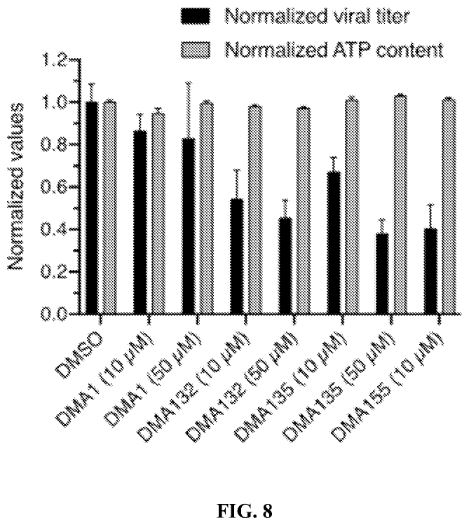

Antiviral activity of the three most active DMA leads (DMA-132, -135, and -155) was confirmed using Vero E6 cells infected with wild-type SARS-CoV-2. Small molecule treatment was performed at 10 μM and 50 μM, respectively. DMA-132 and -135 showed dose-dependent reduction in viral titer compared to DMSO, as measured by median tissue culture infectious dose (TCID₅₀) assay, without measurable effect on cellular viability as measured by ATP content (FIG. 8). Viral titer and cellular content of ATP (CellGro) were normalized to account for the expected variance of raw viral titer between experiments. Notably, all three DMA-132 -135, and -155 have improved antiviral activity when compared to DMA-01, the parent scaffold, thereby corroborating the potential for synthetic tunability of DMAs for SARS-CoV-2 targeting.

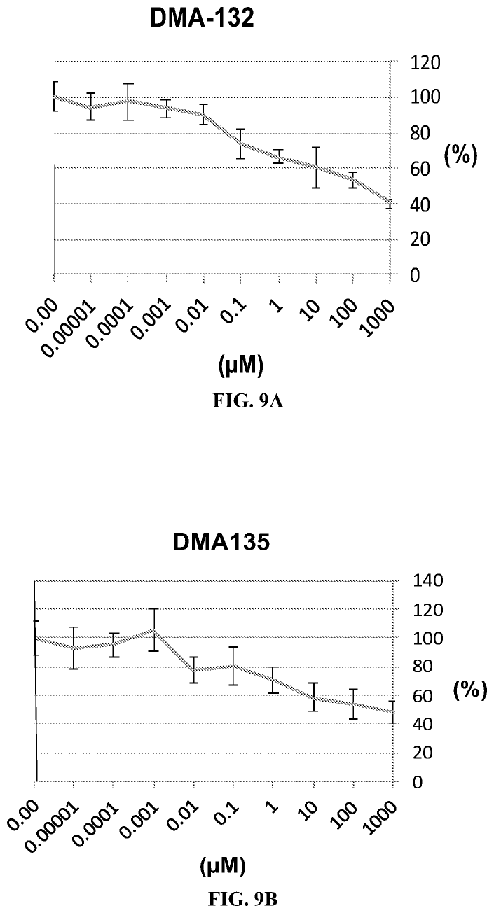

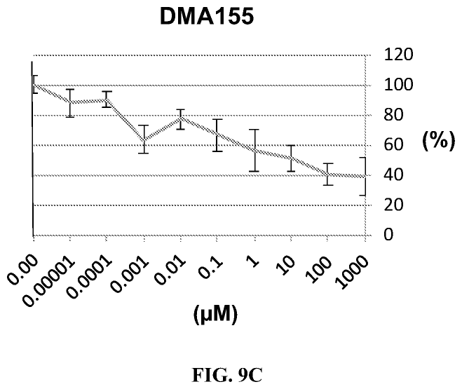

DMA-132, -135, and -155 were tested for longer term (96 hour) toxicity in Vero E6 cells. Small molecules do not significantly reduce cell viability <10 μM, supporting a potential therapeutic window, though more extensive cytotoxicity studies are warranted. In particular, FIGS. 9A-9C show the CC₅₀ of DMA-132 and -135 in Vero E6 cells were >100 μM. CC₅₀ of DMA-155 was about 90 μM, so only 10 μM antiviral data was collected.

### Example 4

### Small Molecule Activity against CoV-2-Luciferase Reporter Gene Expression

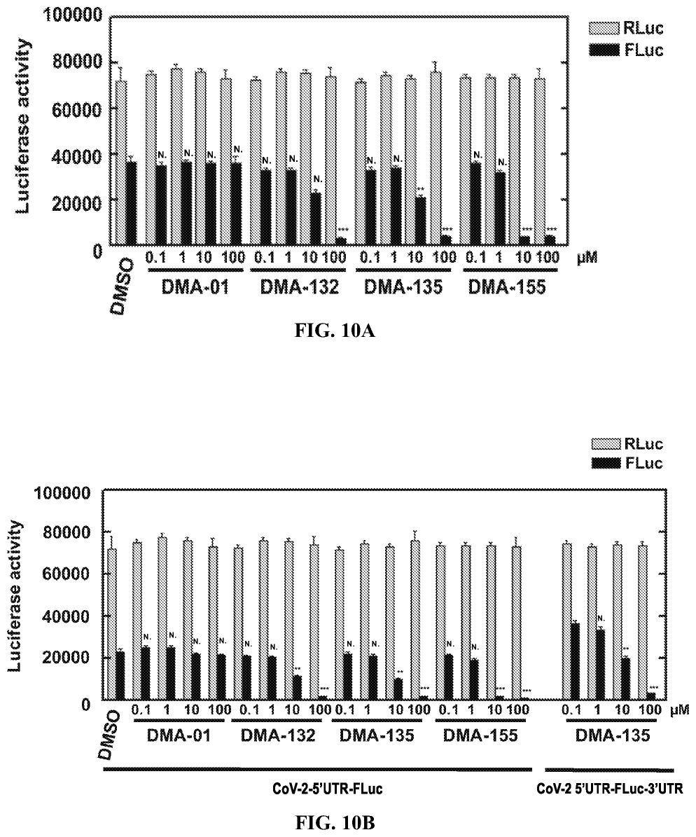

To assess the effect of DMAs on reporter gene expression directed by SARS-CoV-2 sequence elements, a reporter plasmid, pCoV2-5′UTR-FLuc-3′UTR was used as template for in vitro synthesis of CoV-2-5′UTR-FLuc-3′UTR RNA (FIG. 10A). This plasmid contains the 5′-end 805 nucleotide (nt) segment from the virus genome and the 3′-UTR. Thus, the 805 nt segment spans the genomic RNA 5′-UTR (SL 1-5) and ORFla encoding a portion of nsp1 (including SL 6-8) fused in-frame with the firefly luciferase ORF. Plasmid pRL was used as template for the synthesis of control Renilla luciferase reporter RNA lacking SARS-Cov-2 sequences. The RNAs were co-transfected and various concentrations of DMAs were added with the transfections. Two days after transfection, RLuc and FLuc activities were measured using a dual-luciferase reporter assay. As shown in FIG. 10A, addition of 10 μM DMA-132 or -135 reduced FLuc activity, which is under the control of SARS-CoV-2 5′-end and 3′UTR, by approximately 50%. Addition of 10 μM DMA-155 resulted in the largest decrease of FLuc activity, specifically FLuc signal was reduced by ˜90%. A similar level of FLuc signal reduction was achieved in the presence of 10-fold higher concentration of DMA-132, -135 when compared to DMA-155. Activities of the control RLuc remained relatively constant for all DMAs across all concentrations tested; this control indicates that decreases in FLuc required SARS-CoV-2 sequence elements and were not due to any putative cytotoxic effects of DMAs or to non-specific effects on translation. To test whether the CoV-2 3′-UTR contributes to DMA-mediated translational repression, the experiment was repeated using a FLuc reporter, CoV-2-5′UTR-FLuc RNA, in which the CoV-2 3′-UTR was replaced with vector-encoded sequence; thus, the CoV-2 5′-ends of both FLuc reporter RNAs are the same (FIG. 10B). The effects of DMAs on translational repression were virtually identical to those in FIG. 10A. It was noted that select DMAs that inhibited SARS-CoV-2 5′-end-dependent FLuc translation had little effect on EV71 5′UTR-dependent FLuc translation. Thus, the DMAs act at the level of the SARS-CoV-2 5′-end and not at the level of putative structure present in the FLuc coding region. Taken together, these results clearly demonstrate that DMA-dependent suppression of SARS-CoV-2 luciferase reporter activity requires only 5′-end sequences of the virus.

### Example 5

### NMR Profiling and in vitro Affinity of 5′-Stem Loop-DMA Interactions

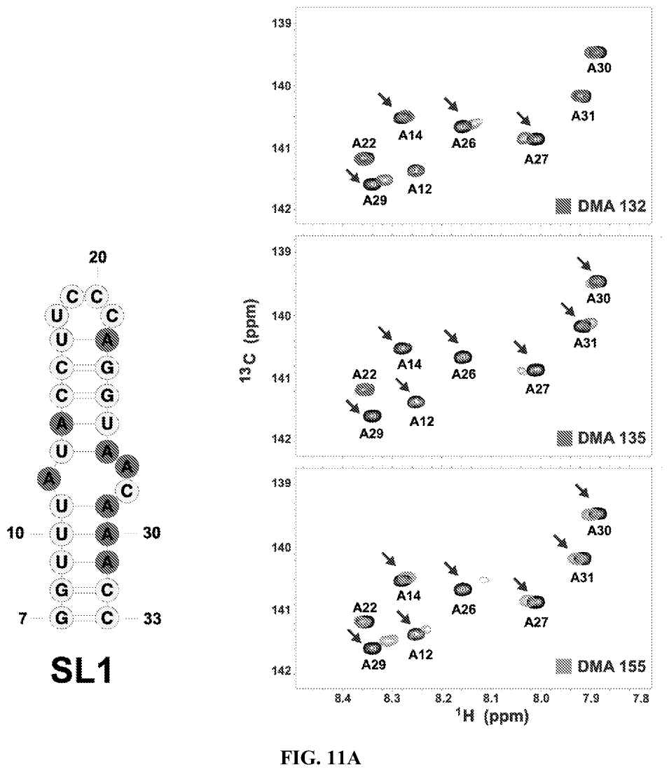

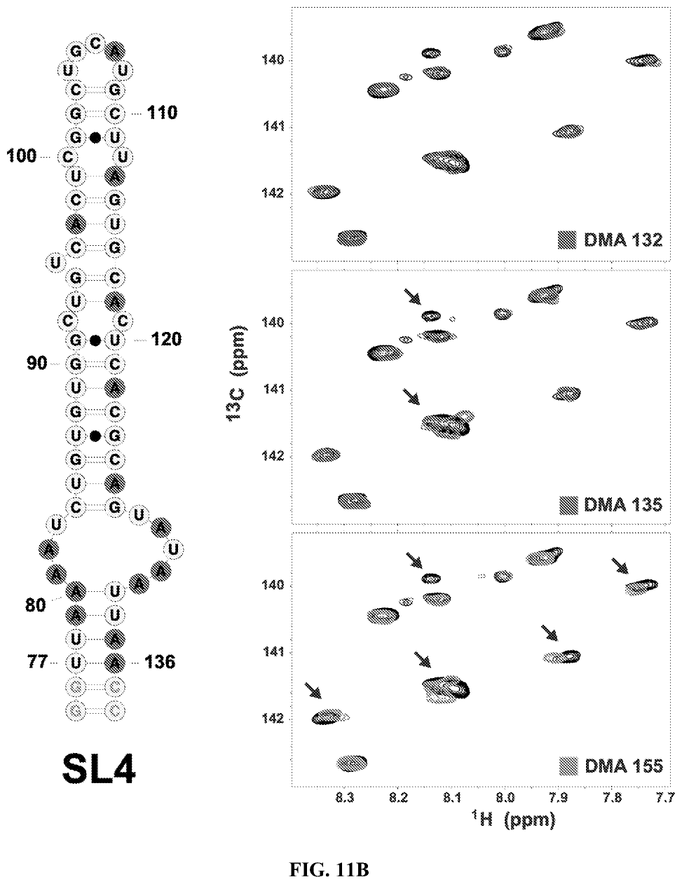

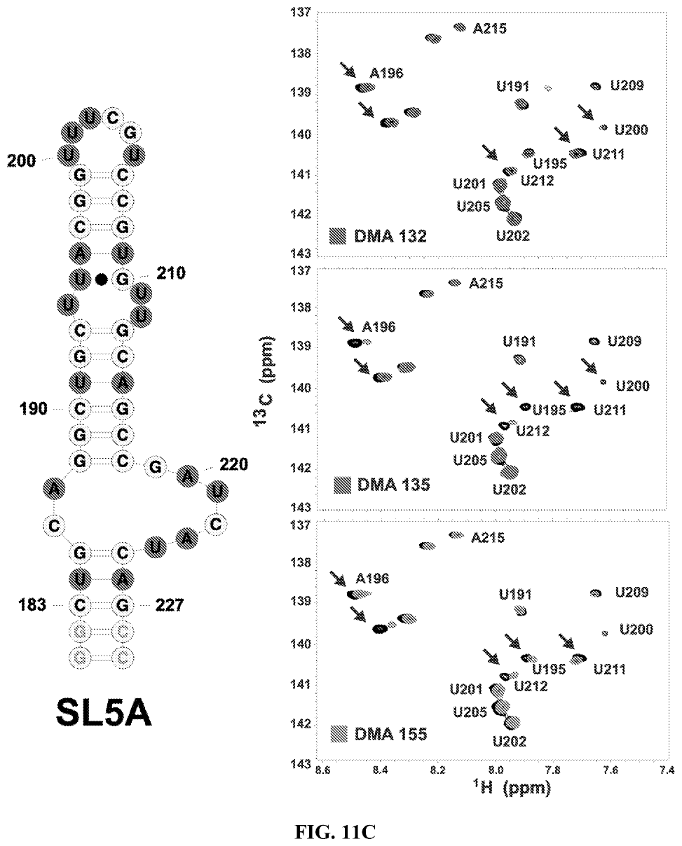

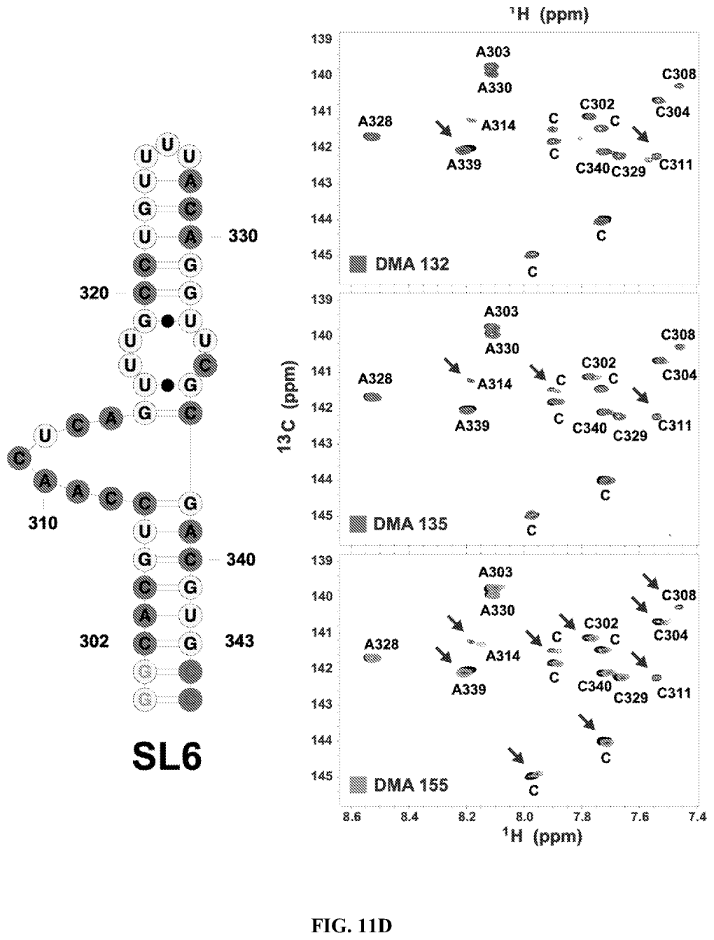

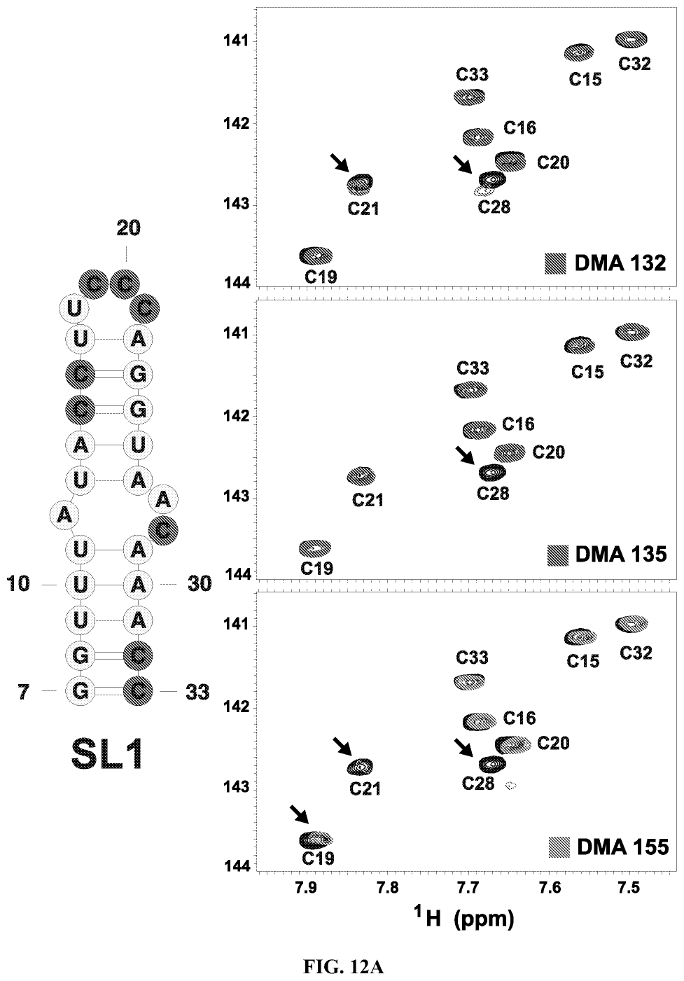

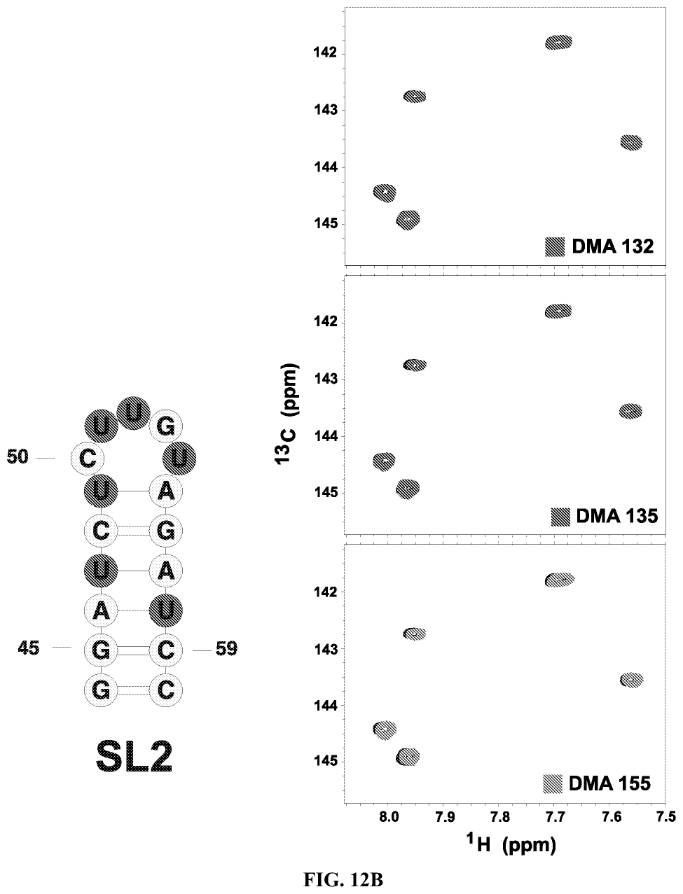

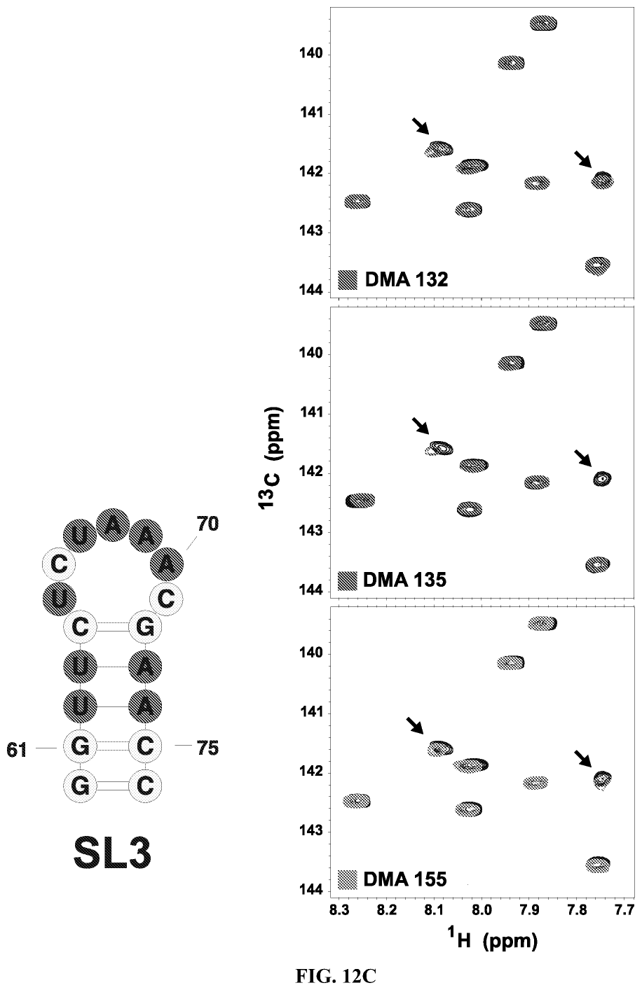

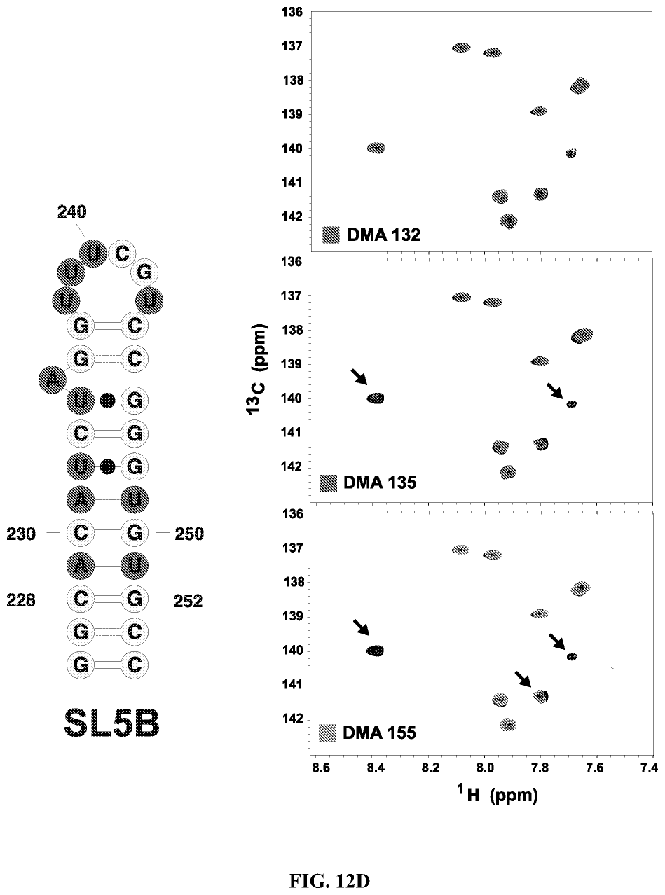

Towards understanding potential mechanisms by which the DMAs inhibit SARS-CoV-2 replication, single-point ¹³C-¹H TROSY HSQC titrations of DMA-132, -135 and -155 into stem loops 1-6 (SL1-6) of the 5′-region were carried out. This region of the 5′-UTR to NMR profile was selected following the reasoning that it contains SLs with assigned functions that can be prepared at sizes amenable to facile NMR screening, shows a degree of selective pressure to maintain sequence and structure, and contains several non-canonical RNA elements that were hypothesized as good targets for the DMAs. An advantage of using ¹³C-¹H TROSY HSQC titrations to profile small molecule-RNA interactions is that the extent of NMR signal perturbation provides a convenient proxy on the degree of binding specificity, even in the absence of chemical shift assignments. The designs of the individual SLs were based on the reasoning that DMAs likely bind the 5′-region stem loops at non-canonical structural elements such as bulge, internal or apical loops. Therefore, isolated SL domains were in vitro transcribed using ¹³C/¹⁵N rNTPs that would maximize NMR signal detection for the non-canonical elements over the base paired regions (FIG. 11A-11D and FIGS. 12A-12D). For example, SL1 was prepared separately as A(¹³C/¹⁵N)- and C(¹³C/¹⁵N)-selectively labeled constructs because adenosines and cytosines are the most abundant nucleobases within or proximal to its internal and apical loops, respectively. Using this strategy, each 5′-region SL was efficiently profiled to determine if the DMAs bind with reasonable affinity and specificity as determined by qualitative assessment of the DMA-induced NMR signal perturbation. FIGS. 11A-11D and FIGS. 12A-12D summarize the effects that the addition of excess (5:1) DMAs have on the NMR spectra of each major SL domain. First, it was observed that the DMAs induced differential chemical shift perturbations (CSPs) or line broadening (LB) for each SL, and the largest signal perturbations (CSPs or LB) were observed in spectra recorded for SLs 1, 4, 5a and 6 (FIGS. 11A-11D). The assignments of SLs 1 and 5a have been previously reported (Richter et al., Biomolecular NMR Assignments, 15, 467-474 (2021); Schnieders et al., Biomolecular NMR Assignments 15, 203-211 (2021)). Each of these 5′-domains contain bulges and/or other internal loops. Second, only a subset of the ¹³C-¹H correlation peaks is perturbed in the spectra providing initial evidence that the DMAs interact through specific surfaces rather than delocalized binding. Third, the extent of the signal perturbations is also differential between DMAs, with some DMAs inducing shifts of the correlation peaks to new positions within the spectra of a given SL and others inducing severe line broadening of the NMR signals. These variable signal perturbations suggest that the DMAs interact with different binding affinities and binding modes. None of the DMAs caused significant changes to spectra recorded on SL2, which contains a 5-nt CUUGU apical loop but no internal loops or bulges. Taken together, the single-point ¹³C-¹H TROSY HSQC titrations provide compelling evidence that the DMAs make specific interactions with the SARS-CoV-2 5′-region via surfaces composed of non-canonical structural elements.

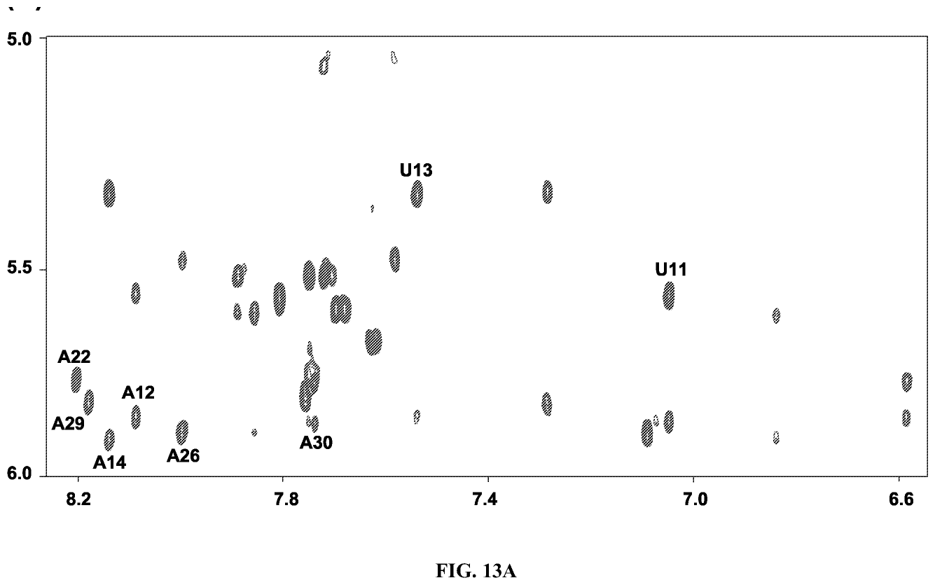

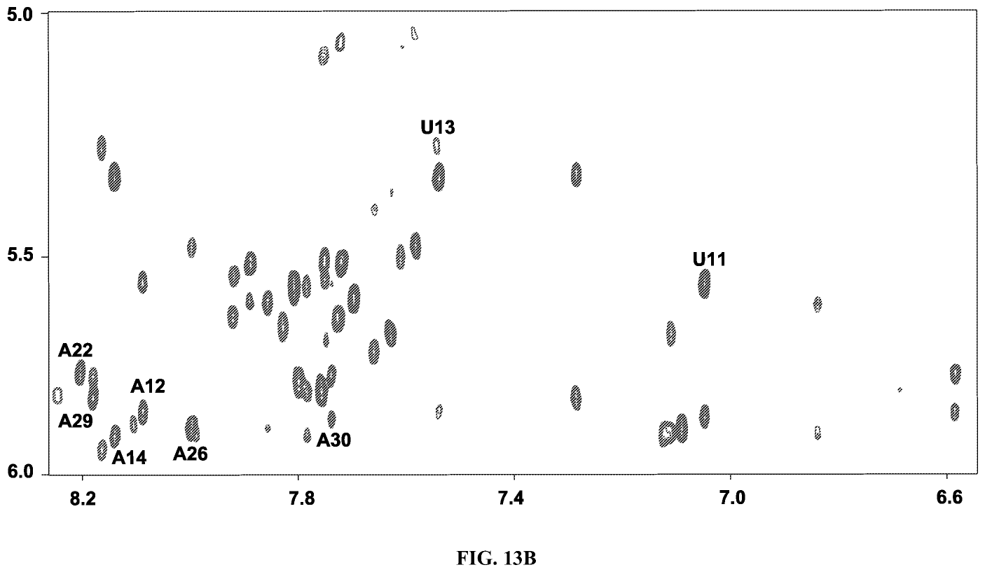

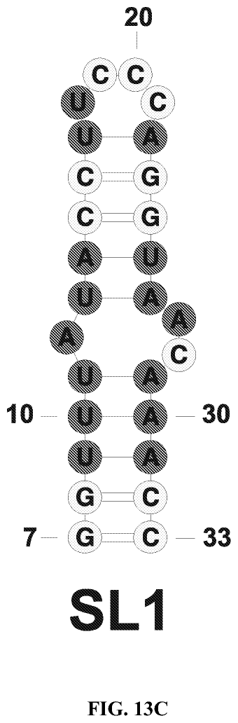

Notably, SL1 and SL6 experienced the most significant NMR signal perturbations upon addition of excess DMAs (FIG. 11A and FIG. 11D). NMR signal perturbations are observed at other SLs but to a lesser degree based on our selection criteria. For SL1, addition of DMAs 132 and 155 induced the migration of several correlation peaks to new spectral positions (FIG. 11A). By comparison, addition of DMA-135 induced severe line broadening of ˜50% of the total observed correlation peaks recorded for the A(¹³C/¹⁵N)- and C(¹³C/¹⁵N)-selectively labeled SL1 constructs (FIG. 11A and FIGS. 12A-12D). In the presence of 5-fold excess DMA-135, most of the NOE stacking patterns disappear for a CG(²H), AU(²H_{3′-5″}-)-selectively labeled SL1 construct, whereas significantly more NOE cross peaks are still observed for the 5:1 (DMA-155)-SL1 complex (FIG. 13A-13C). These differences likely reflect binding heterogeneity and collectively suggest that DMAs 132, 135 and 155 differentially interact with SL1 despite having similar core scaffolds (FIG. 11A).

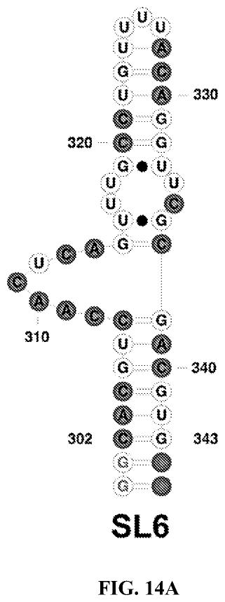

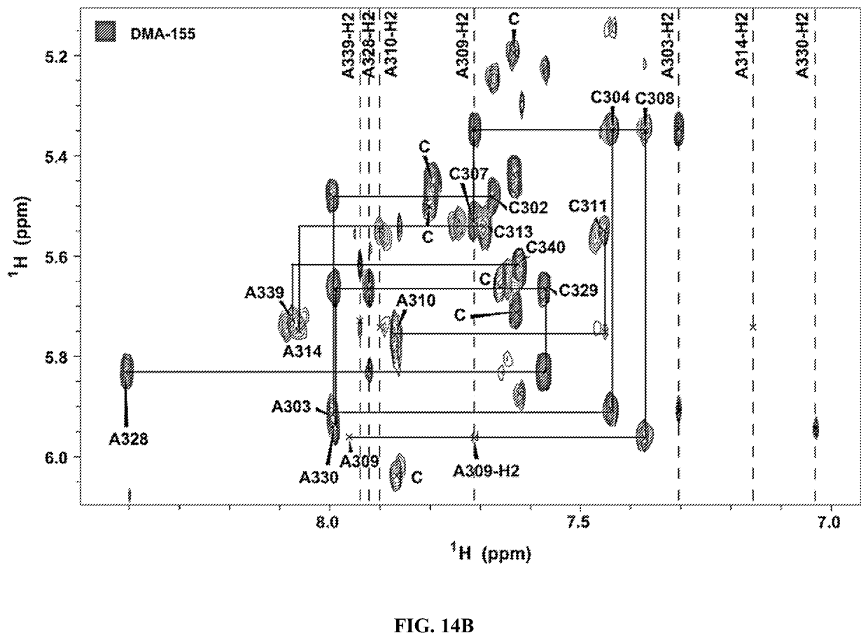

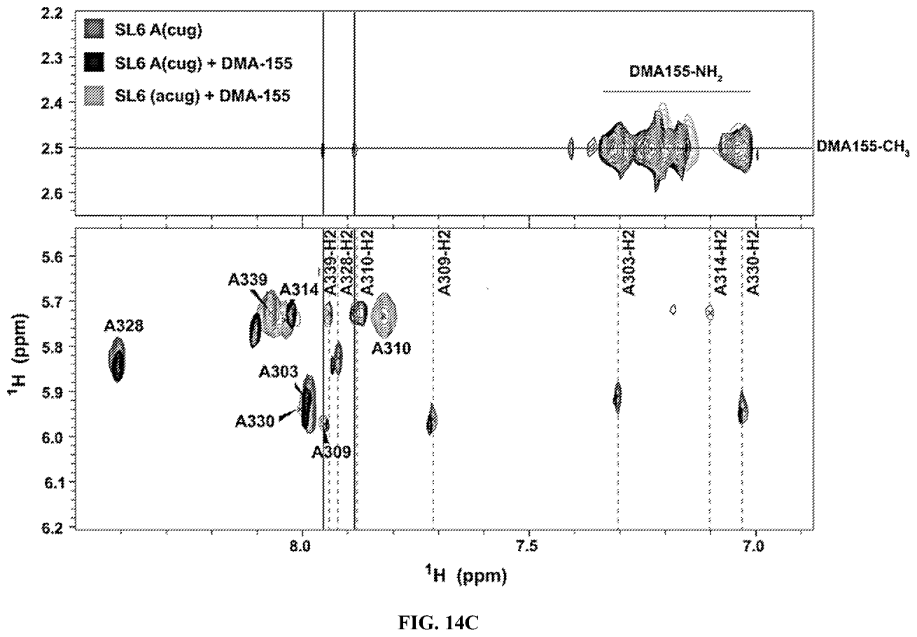

For SL6, addition of excess DMA-155 induced a combination of CSPs and LB to a greater extent than DMA-132 or DMA-135. To further assess the nature of these signal perturbations on the SL6 structure, a ¹H-¹H NOESY (tₘ=250 ms) spectrum was recorded with UG(²H), AC(²H_{3′-5″})-selectively labeled SL6 constructs, which was chosen based on the abundance of A and C residues in the bulge (FIG. 14A). FIG. 14A shows the ¹H-¹H NOESY spectra of free SL6 overlaid with its complex to which a 5:1 molar ratio of DMA-155 was added. Based on the intra-NOEs observed between A and C residues of SL6, tentative chemical shift assignments were determined (FIGS. 14B and 14C). The addition of excess small molecule caused CSPs to A339H8, A339H2, A310H8, A310H2, C311H6, and A314H8 spin systems, which are located within or in proximity to the bulge. This supports a model in which DMA-155 preferentially interacts with the bulge region of SL6. Notably, the presence of intra-NOEs in the complex indicates the preservation of base stacking within the (DMA-155)-SL6 complex. In addition, the 1H-¹H NOESY spectra of CUG(²H), A(²H_{3′-5″})-selectively labeled SL6 overlaid with its DMA-155 bound form (FIG. 14C) show inter-NOEs between the methyl protons of DMA-155 (2.5 ppm) and both A339H2 (7.94 ppm) and A310H2 (7.88 ppm), though these particular inter-NOEs are weak. As further evidence of the involvement of A339 and A310 in binding, their H8 spin systems also showed CSPs upon the addition of DMA-155. Interestingly, four additional broad NOE peaks appeared between the methyl protons and protons with chemical shifts between ˜6.5-7 ppm (FIG. 14C). It was reasoned that these might be intra-NOEs between the methyl groups and amino group located on the central six-membered ring system. To further investigate the origin of these NOEs, a ¹H-¹H NOESY spectrum of ACGU(²H)-labeled SL6 bound to DMA-155 (shown in orange, FIG. 14C) displayed identical broad NOE peaks. This shows that these peaks signify intra-NOEs within DMA-155 itself, which do not appear in the ¹H spectrum of the free small molecule in D₂O but only in the environment of the SL6 complex. The possibility of these peaks arising from inter-NOEs between the amino of the RNA and methyl protons was eliminated based on ¹⁵N-HSQC NMR experiments of DMA-155 titrated into ¹⁵N-fully labeled SL6. In sum, through the support of intra- and inter-NOEs, DMA-155 shows a degree of specificity to the bulge region of SL6.

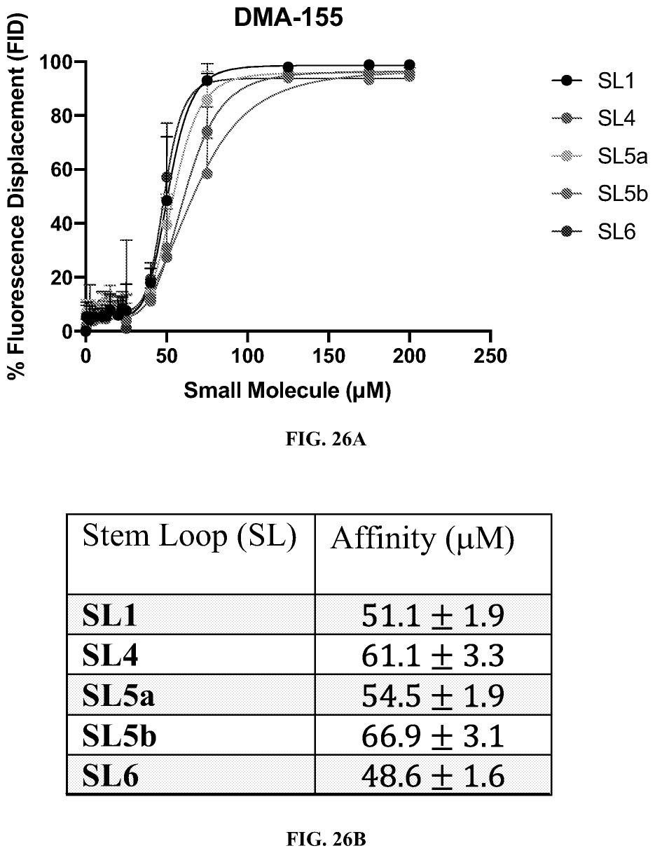

Moreover, the affinity of the three leads for the stem loop constructs was investigated via in vitro indicator displacement assays (IDA) (Davila-Calderon et al., Nature Communications 11, 4775 (2020); Wicks, Methods 167, 3-14 (2019); Patwardhan, et al., Org Biomol Chem 17, 1778-1786 (2019)). DMA-155 was revealed to be a strong binder with highest affinity for SL6, in agreement with NOE studies (FIGS. 14A-14C and FIGS. 26A-26B). DMA-135 showed weak affinity for SL1, SL4, and SL6; the parent scaffold (DMA-001) and DMA-132 did not show binding affinity against any of the constructs under these conditions. The measured displacement of the indicator (CD₅₀) in vitro is about 10-fold weaker than the IC₅₀ recorded via luciferase assays, which may highlight the differences between these assays. CD₅₀ measurements depend on both the affinity and binding mode of the indicator used, while in cell assays reflect binding in the presence of a larger RNA context and in the presence of proteins. Additional biophysical studies are underway to better understand the relationship between these in vitro and in cellulo assays.

### Example 6

### In silico Ligand Screening of DMA Focused Library against 5′-UTR and Adjacent RNA Structures

To generate potential 3D models of each SL, Fragment Assembly of RNA with Full-Atom Refinement (FARFAR) was used. FARFAR was chosen to generate preliminary models as it has consistently been demonstrated to be the most accurate RNA 3D prediction algorithm (Miao et al., Rna 23, 655-672 (2017); Rangan and Zheludev, RNA, 26, 937-959 (2020)). Between 5,000 and 100,000 models were generated for each SL and then ˜15 representative clusters were generated.

The lowest energy conformation from each cluster was used to generate an ensemble for each SL that was submitted to ICM pocket finder to find and characterize possible binding pockets (see, Table 1).

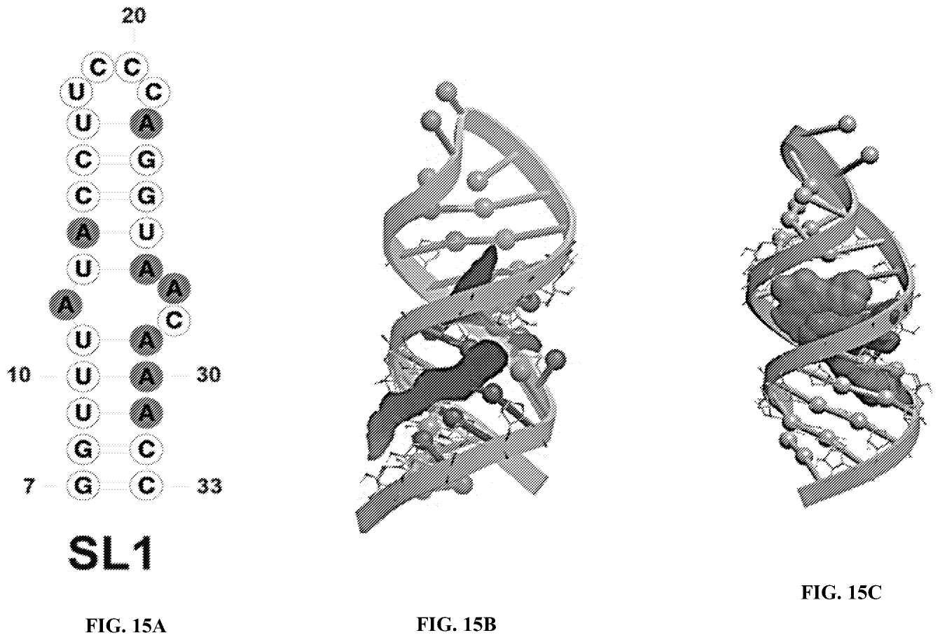

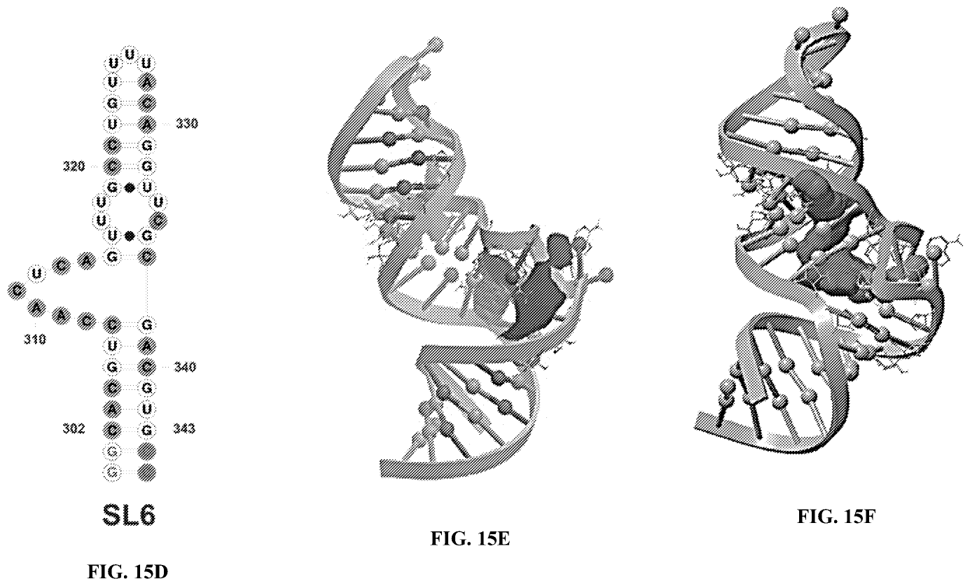

Notably, SL2, which showed no change in 18C-¹H HSQC NMR chemical shifts upon small molecule addition, did not have any identifiable binding pocket. SLs 1, 3, and 5b presented binding pockets with low to intermediate scores in terms of volume, area, hydrophobicity, burriedness and druggability score (DLID) parameters often used to describe a binding pocket's fitness. Notably, SL3 and 5b presented minor chemical shift perturbations upon small molecule addition. ¹³C-¹H TROSY HSQC NMR experiments showed significant changes upon small molecule binding to SL1, 5a, and SL6 (FIGS. 11A-11D), all of which had the highest scores in all parameters (FIG. 15A-15F). Most importantly, the structure that presents the highest chemical shift perturbations upon binding to the small molecule leads, SL6, is the only SL to have a binding pocket with positive DLID score (0.45, see, Table 1). DIM score has been found as a good predictor of druggability of protein binding pockets but often presents highly negative scores in RNA due to the charged backbone of RNA (Hewitt et al., Bioorg Med Chem 27, 2253-2260 (2019); Sheridan et al., J Chem InfModel 50, 2029-2040 (2010)). Overall, this binding pocket analysis strongly correlates with NMR experimental data.

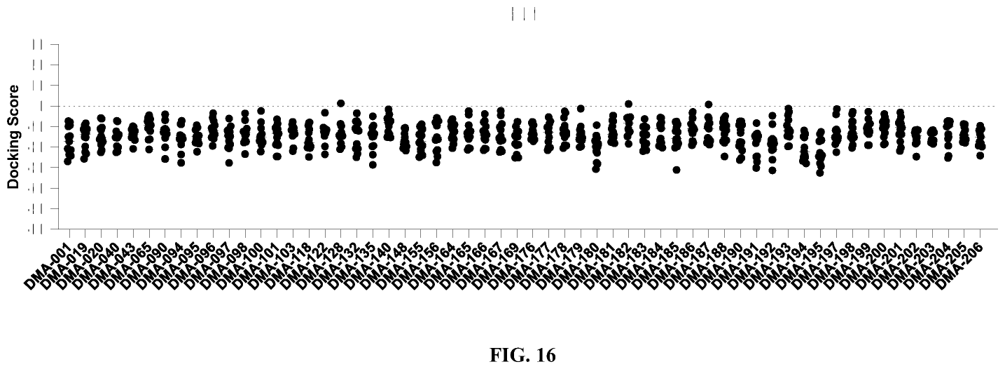

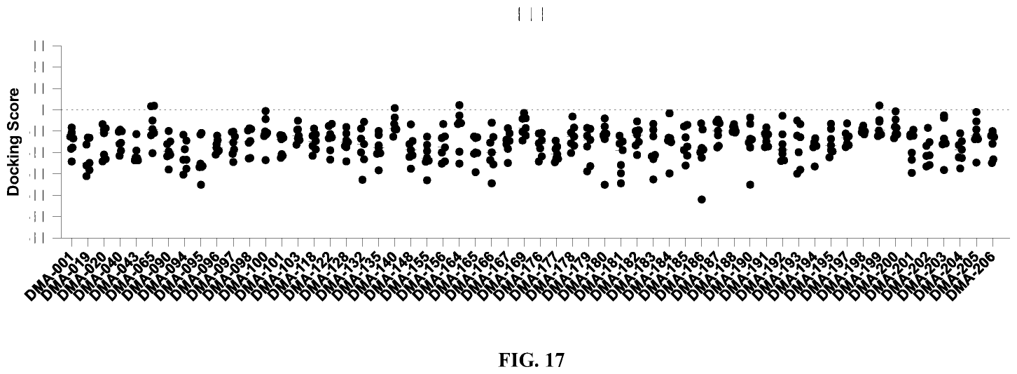

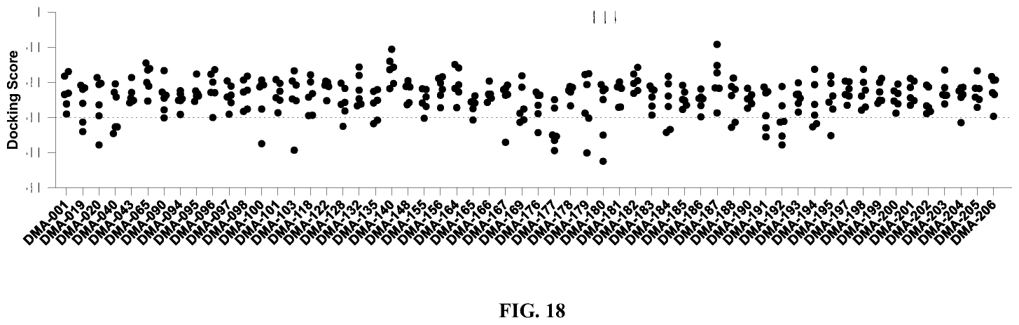

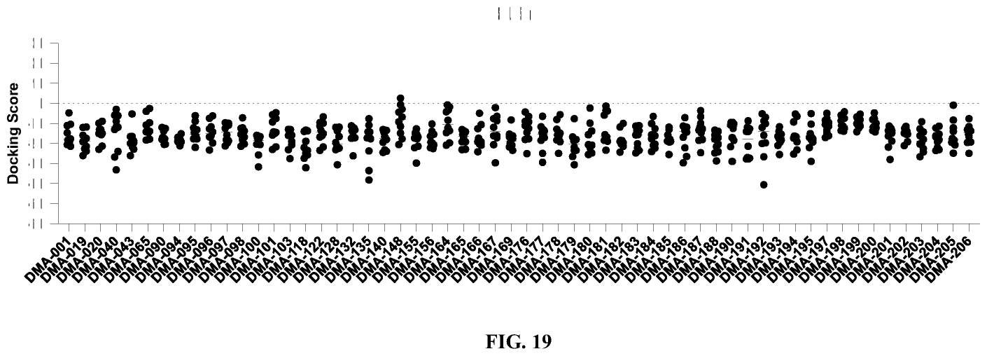

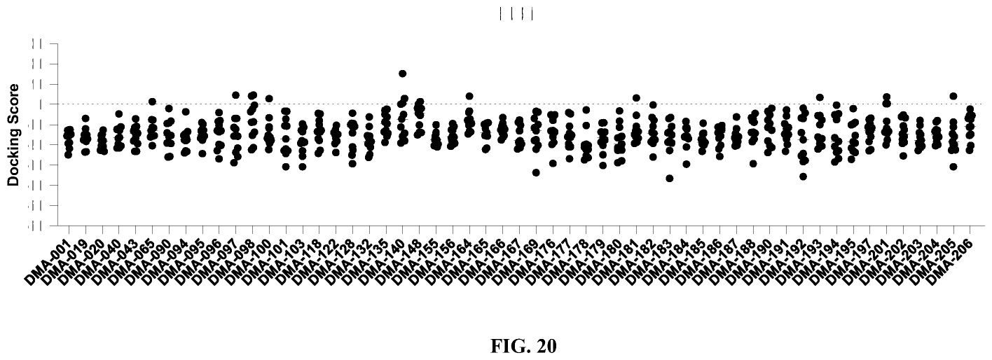

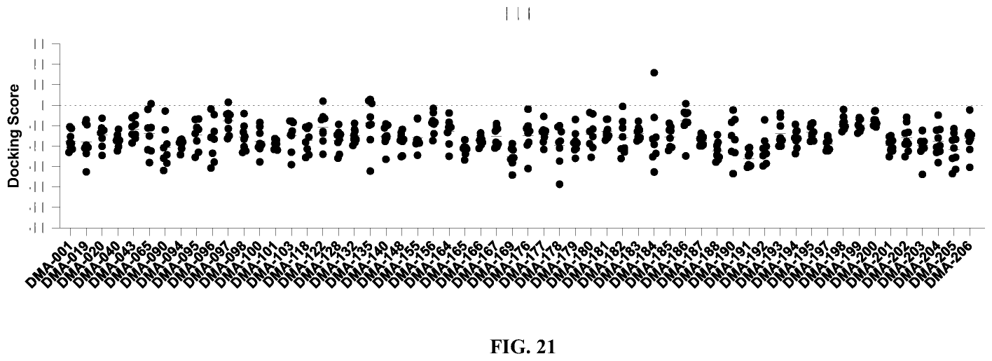

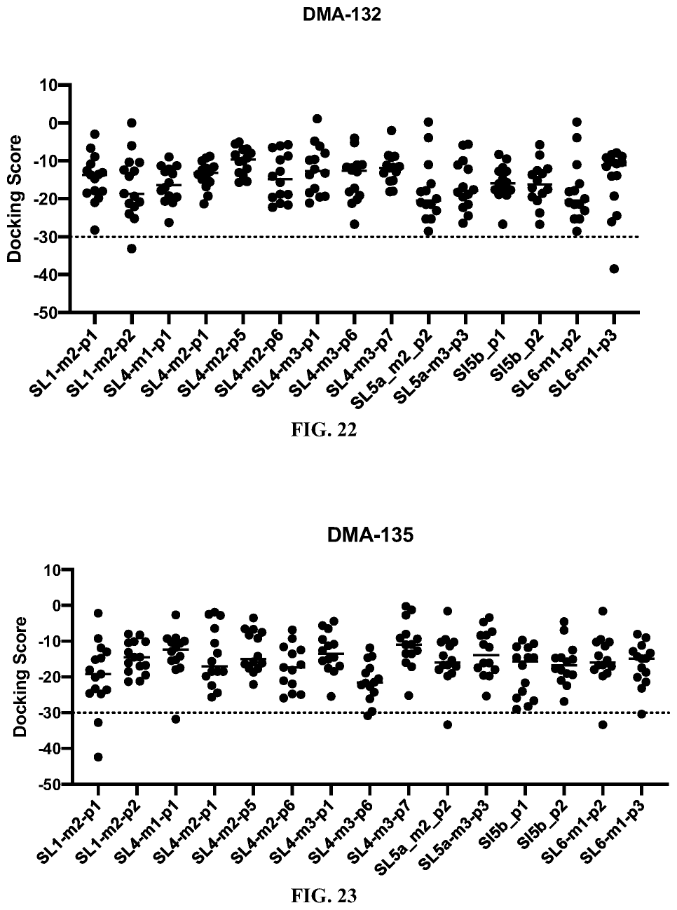

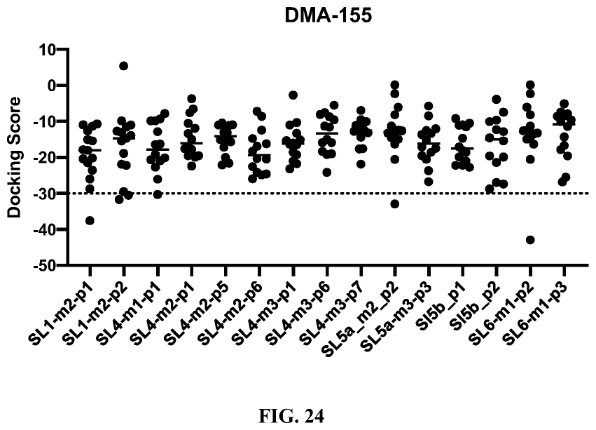

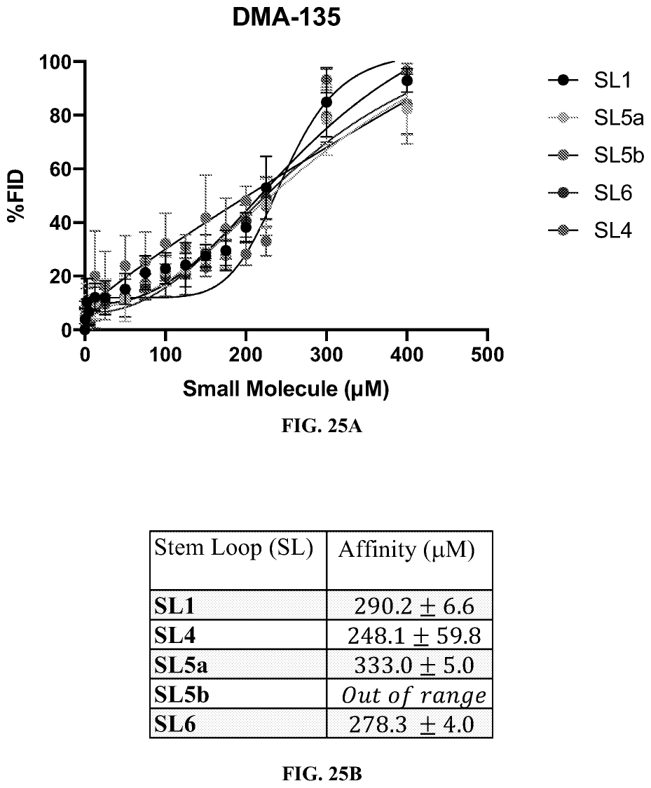

Previously published 55 member DMA library was then docked against the clustered FARFAR-generated stem loop structures (FIGS. 16-21). In line with the results from the pocket analysis and NMR experimental data, the overall number of hits across constructs is highest for SL1, SL4, SL5a, and SL6, corroborating their potential as therapeutic targets. Hit ligands DMA-132, 135, and 155 were among the top predicted binding ligands. Furthermore, if the docked structures are limited to the sub-library of DMAs tested against OC43 and SARS-CoV-2, the three hit ligands score in the top 5 of every stem loop. Notably, when analyzing the binding location that yielded the best docking scores of the three hit molecules against SL6, it was noted that while DMA 135 and 155 bound best in one binding pocket (FIG. 15F in blue), DMA 132 docks best in the adjacent binding pocket (FIG. 15F in red). When comparing the NMR profiles of the three small molecules, it was noted that DMA 135 and 155 have a similar perturbation pattern, while DMA 132 has a slightly different profile, suggesting a possible different binding mode. To further investigate this hypothesis, FARFAR 3D models were generated for every individual bulge and apical loop motif to increase the conformational diversity at these sites within the SL clusters, which are otherwise largely dictated by the respective positions of the double stranded regions. The DMA library was then rescreened against each of the clustered motifs. The three small molecule leads DMA-132, DMA-135, and DMA-155 consistently scored among the top hits (FIGS. 17-21). Furthermore, each of the molecule reported the best docking score for SL1, SL5a, and SL6 and, more specifically, for motifs created for internal bulge motifs (FIGS. 22-24). In the case of DMA-135, for example, the best docking score was found for the internal loop motif of SL1, in agreement with HSQC data that reported DMA-135 as the best binder for SL1 (FIGS. 25A-25B). Analogously, DMA-155 had the best docking score for the internal loop pocket of SL6, in line with NOE data that identified DMA-155 as the best and most specific binder for SL6 (FIGS. 26A-26B). While preliminary, this docking analysis corroborates the NMR data and the fitness of these ligands for SARS-CoV-2 5′-stem loop targeting while also supporting its utility as a tool in the identification of new SARS-CoV-2 RNA-targeting ligands.

Discussion: Screening of synthetic RNA-focused libraries in recent years has provided the field with many RNA-binding bioactive small molecules and some of the highest hit rates among small molecule screening approaches (Morgan et al., Nucleic Acids Research 46, 8025-8037 (2018)). Amiloride, a known RNA-binding scaffold, has been synthetically tuned for a range of RNA secondary structures and recently yielded a novel antiviral lead for the treatment of enterovirus 71. In this case, amiloride inhibited viral translation by binding the viral 5′-UTR and modulating RNA:host protein interactions. SARS-CoV-2 also contains a highly conserved 5′-end that is reported to play a crucial role in viral replication and hijacking of host cell translational machinery. The presence of multiple bulge or internal loops, the secondary structural elements that amilorides have been reported to bind most effectively, makes the 5′-UTR and the adjacent SL6 ideal therapeutically relevant targets for small molecule probing. An initial DMA focused library screen against OC43-infected Vero E6 cells allowed for the identification of three lead compounds, namely DMA-132, -135, and -155 that significantly reduced viral titer. Initial structure-activity relationships could also be resolved, highlighting the critical substitution of the dimethylamine group at the C5 position and rigid aromatic substituents at the C6 position. These molecules were also found to be active against SARS-CoV-2 in both screening-format qRT-PCR and infectious viral titer assays. Luciferase assays revealed the presence of the 5′-UTR and proximal region as necessary and sufficient for translation inhibition upon small molecule treatment. Markedly, all in vitro assays identified DMA-155 as the strongest SARS-CoV2 inhibitor, with the largest reduction of luciferase signal and highest decrease in viral titer at 10 μM.

NMR profiling of leads DMA-132, -135 and -155 against each of the major 5′-UTR and adjacent stem loop domains revealed that the DMAs bind preferentially to SLs that contain large internal or bulge loops. Interestingly, SL1's NMR data revealed CSPs and LB with DMA-135, which signifies its binding onto a specific surface with a disruption in stacking interactions, unlike DMA-155. Notably, SL6, which contains a moderately conserved and weakly paired ˜16-nt bulge loop, showed one of the most significant CSPs when titrated with DMAs. Specifically, NOE experiments revealed that DMA-155 binds specifically to SL6, a finding that was corroborated by in vitro indicator displacement assays (IDA), which identified SL6 as the preferred target for DMA-155. Interestingly, the 5′-side of the SL6 bulge loop has been proposed to interact with the SARS-CoV-2 Nucleocapsid (N) protein under phase separation conditions and may also impact genome packaging (Iserman et al., bioRxiv, 2020.2006.2011.147199 (2020)). The modulation of the N protein:SL6 interaction is to be investigated as a potential antiviral mechanism in future work.

Finally, in silico analysis corroborated the experimental trend observed with NMR experiments, identifying some of the stem loops with predicted bulges (SL1, SL4, SL5a, SL6) as those with binding pockets with highest druggability. The small molecules that showed highest antiviral activity, namely DMA-132, -135, and -155 scored highest in the stem loops that reported significant chemical shift perturbations upon small molecule binding. Additionally, refined docking of the three small molecule leads against motif-specific clusters revealed SL1 to be the preferred target for DMA-135 and SL6 as the favored target for DMA-155, in line with NMR and IDA results.

In conclusion, the present disclosure identified drug-like small molecules that reduced SARS-CoV-2 replication and were the first antivirals to target the conserved RNA stem loops in the 5′-end region of SARS-CoV-2. Work is underway to further characterize the mode of action of these ligands, particularly putative impacts on RNA:protein interactions and specific steps in the viral replication cycle. Indeed, these leads are uniquely poised to further elucidate the relationship between in vitro preferential binding and small molecule mediated SL-specific alterations of virus:host interactions. These small molecules offer the opportunity to understand the contribution of individual 5′-end stem loops to viral proliferation in a system where mutational studies are difficult due to genome size. Once characterized, it is expected that these amiloride-based ligands to serve as chemical biology tools to help understand CoV RNA molecular biology, such as N-dependent genome packaging and other cellular stages of the viral RNA replication process. Importantly, an efficient framework has been established to identify novel RNA-targeted CoV antivirals that will serve not only the SARS-CoV-2 pandemic but future coronavirus pandemics.

One skilled in the art will readily appreciate that the present disclosure is well adapted to carry out the objects and obtain the ends and advantages mentioned, as well as those inherent therein. The present disclosure described herein is presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

What is claimed is:

1. A compound of Formula (I), a pharmaceutical acceptable salt, a hydrate, a prodrug, an ester, or a derivative thereof: 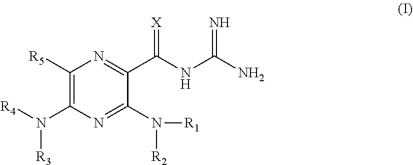
wherein R₁ and R₂ are independently H, C₁-C₈ alkyl, substituted C₁-C₈ alkyl, C₁-C₈ alkenyl, substituted C₁-C₈ alkenyl, C₃-C₆ substituted cycloalkyl, a C₃-C₆ unsubstituted cycloalkyl, aryl, or substituted aryl;
R₃, and R₄ are independently H, C₁-C₈ alkyl, substituted C₁-C₈ alkyl, C₁-C₈ alkenyl, substituted C₁-C₈ alkenyl, C₃-C₆ substituted cycloalkyl, a C₃-C₆ unsubstituted cycloalkyl, aryl, substituted aryl, or R³ and R⁴ may be taken together to form a C₃-C₆ substituted cycloalkyl, a C₃-C₆ unsubstituted cycloalkyl, C₃-C₆ substituted cycloalkenyl, a C₃-C₆ unsubstituted cycloalkenyl, a substituted heterocycle, a unsubstituted heterocycle, or a C₃-C₂₄ heteroaromatic;
R₅ is C₁-C₈ substituted alkyl, C₁-C₈ unsubstituted alkyl, C₃-C₆ substituted cycloalkyl, C₃-C₆ unsubstituted cycloalkyl, a substituted heterocycle, an unsubstituted heterocycle, C₃-C₂₄ aromatic, C₃-C₂₄ heteroaromatic, 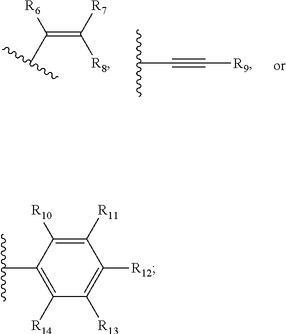
and
R₆, R₇, R₈, R₉, R₁₀, R₁₁, R₁₂, R₁₃, and R₁₄ independently are H, Cl, Br, F, CF₃, C₁-C₆ substituted alkyl, C₁-C₆ unsubstituted substituted alkyl, OCH₃, NH₂, NHCH₃, N(CH₃)₂, CN, cyclopropyl, cyclobutyl, cyclopentyl, cyclohexyl, or phenyl; and
X is O, S, or NH.

2. The compound of claim 1, wherein R₁ and R₂ independently are H, C₁-C₄ alkyl, C₁-C₄ substituted alkyl, C₁-C₄ alkenyl, C₃-C₅ substituted cycloalkyl, a C₃-C₅ unsubstituted cycloalkyl, aryl, or substituted aryl;
R₃, and R₄ are independently H, C₁-C₄ alkyl, C₁-C₄ substituted alkyl, C₁-C₄ alkenyl, C₃-C₅ substituted cycloalkyl, a C₃-C₅ unsubstituted cycloalkyl, aryl, substituted aryl, or R³ and R⁴ may be taken together to form a C₃-C₅ substituted cycloalkyl, a C₃-C₅ unsubstituted cycloalkyl, C₄-C₁₄ heteroaromatic, a substituted heterocycle, or an unsubstituted heterocycle;
R₅ is C₁-C₄ substituted alkyl, C₁-C₄ unsubstituted alkyl, C₃-C₅ substituted cycloalkyl, C₃-C₅ unsubstituted cycloalkyl, C₄-C₁₄ aromatic, C₄-C₁₄ heteroaromatic, a substituted heterocycle, an unsubstituted heterocycle, 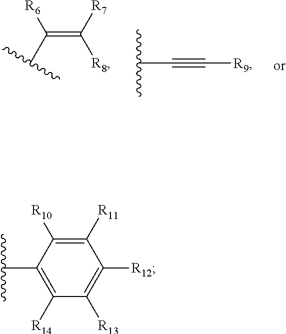
R₆, R₇, R₈, R₉, R₁₀, R₁₁, R₁₂, R₁₃, and R₁₄ independently are H, Cl, Br, F, OCH₃, NH₂, NHCH₃, N(CH₃)₂, or phenyl; and
X is O.

3. The compound of claim 2, wherein R₁, R₂, R₃, and R₄ independently are H, CH₃, CH₃CH₂, CH(CH₃)₂, C(CH₃)₃, or phenyl;
R₅ is 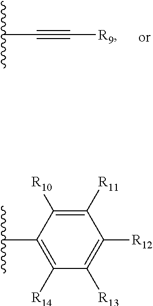
R₆, R₇, R₈, R₉, R₁₀, R₁₁, R₁₂, R₁₃, and R₁₄ independently are H, CH₃, F, CF₃, or phenyl; and X is O.

4. The compound of claim 1, wherein R₁, R₂, R₃, and R₄ are independently H or CH₃;
R₅ is 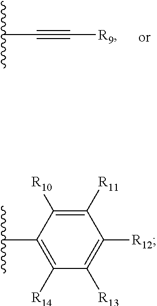
and
R₉, R₁₀, R₁₁, R₁₂, R₁₃, and R₁₄ independently are H or phenyl; and X is O.

5. The compound of claim 1, the wherein R₁ and R₂ are H; R₃, and R₄ are CH₃,
R₅ is 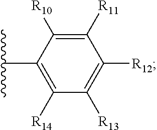
and
R₁₀, R₁₁, R₁₂, R₁₃, and R₁₄ are H; X is O as shown in the compound of Formula (II):

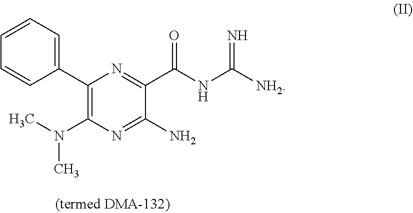 [SMILES: CN(C)c1nc(N)c(C(=O)NC(=N)N)nc1-c1ccccc1]

6. The compound claim 1, wherein R₁ and R₂ are H; R₃, and R₄ are CH₃,
R₅ is 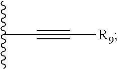
and
R₉ is phenyl; and X is O as shown in the compound of Formula (III):

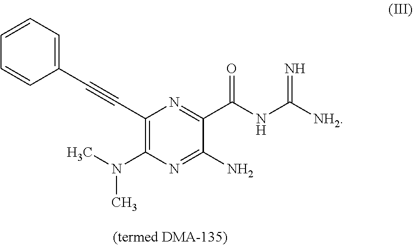 [SMILES: CN(C)c1nc(N)c(C(=O)NC(=N)N)nc1C#Cc1ccccc1]

7. The compound of claim , wherein R₁ and R₂ are H; R₃, and R₄ are CH₃,
R₅ is 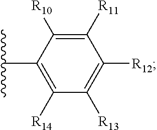
and
R₁₀, R₁₁, R₁₂, R₁₃, and R₁₄ are H; R₁₂ is phenyl; and X is O as shown in the compound of Formula (IV):

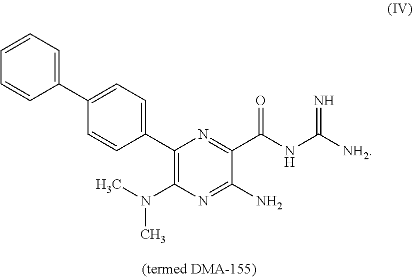 [SMILES: CN(C)c1nc(N)c(C(=O)NC(=N)N)nc1-c1ccc(-c2ccccc2)cc1]

8. A pharmaceutical composition comprising the compound of any one of claims 1-7 and a pharmaceutically acceptable carrier and/or excipient.

9. A method of inhibiting viral replication in a cell, the method comprising administering to the cell an effective amount of the compound of any one of claims 1-7 or an effective amount of the pharmaceutical composition of claim 8.

10. A method of preventing or treating a viral infection in a subject, the method comprising administering to the subject an effective amount of the compound of any one of claims 1-7 or an effective amount of the pharmaceutical composition of claim 8.

11. The method of claim 9 or 10, wherein the virus comprises a coronavirus.

12. The method of claim 11, wherein the coronavirus is selected from the group consisting of 229E (alpha coronavirus), NL63 (alpha coronavirus), OC43 (beta coronavirus), HKU1 (beta coronavirus), MERS-CoV (beta coronavirus), SARS-CoV (beta coronavirus), and SARS-CoV-2, or a combination thereof.

13. The method of claim 12, wherein the coronavirus comprises SARS-CoV-2.

14. A method of preventing or treating COVID-19 in a subject, the method comprising administering to the subject an effective amount of the compound of any one of claims 1-7 or an effective amount of the pharmaceutical composition of claim 8.

15. The method of any one of claims 9-14, wherein the compound or the pharmaceutical composition is administered in conjunction with another antiviral or anti-inflammatory agent.

16. Use of the compound of any one of claims 1-7 or the pharmaceutical composition of claim 8 as an antiviral agent.

17. The use of claim 16, wherein the antiviral agent targets coronavirus RNA.

18. The use of claim 17, wherein the coronavirus is selected from the group consisting of 229E (alpha coronavirus), NL63 (alpha coronavirus), OC43 (beta coronavirus), HKU1 (beta coronavirus), MERS-CoV (beta coronavirus), SARS-CoV (beta coronavirus), and SARS-CoV-2, or a combination thereof.

19. The use of claim 18, wherein the coronavirus comprises SARS-CoV-2.

20. Use of the compound of any one of claims 1-7 or the pharmaceutical composition of claim 8 as an antiviral agent for preventing or treating COVID-19.


SEQUENCE LISTING (7 sequences)

SEQ ID NO: 1 - DNA, 322 bp, Betacoronavirus
attgtgagcgatttgcgtgcgtgcatcccgcttcactgatctcttgttagatctttttgtaatctaaactttataaaaacatccactccctgtaatctatgcttgtgggcgtagatttttcatagtggtgtttatattcatttctgctgttaacagctttcagccagggacgtgttgtatcctaggcagtggcccgcccataggtcacaatgtcgaagatcaacaaatacggtctcgaactacactgggctccagaatttccatggatgtttgaggacgcagaggagaagttggataaccctagtagttcagaggtggatat

SEQ ID NO: 2 - DNA, 348 bp, Betacoronavirus
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgt

SEQ ID NO: 3 - DNA, 347 bp, Betacoronavirus
atattaggtttttacctacccaggaaaagccaaccaacctcgatctcttgtagatctgttctctaaacgaactttaaaatctgtgtagctgtcgctcggctgcatgcctagtgcacctacgcagtataaacaataataaattttactgtcgttgacaagaaacgagtaactcgtccctcttctgcagactgcttacggtttcgtccgtgttgcagtcgatcatcagcatacctaggtttcgtccgggtgtgaccgaaaggtaagatggagagccttgttcttggtgtcaacgagaaaacacacgtccaactcagtttgcctgtccttcaggttagagacgtgctagt

SEQ ID NO: 4 - DNA, 347 bp, Betacoronavirus
atattaggtttttacctacccaggaaaagccaaccaacctcgatctcttgtagatctgttctctaaacgaactttaaaatctgtgtagctgtcgctcggctgcatgcctagtgcacctacgcagtataaacaataataaattttactgtcgttgacaagaaacgagtaactcgtccctcttctgcagactgcttacggtttcgtccgtgttgcagtcgatcatcagcatacctaggtttcgtccgggtgtgaccgaaaggtaagatggagagccttgtccctggtttcaacgagaaaacacacgtccaactcagtttgcctgtcttacaggttcgcgacgtgctcgt

SEQ ID NO: 5 - DNA, 355 bp, Betacoronavirus
cattaaagagaatagcatagctatccctctctctccgttctcttgtagaactcttgttttaacgaacttaattaaaagcccttatactttgtttaggtagtggcactagtgccagacctatgcattgtagtgggtttagtaaaaattaaatttttatttttcatttagagctgcgtgtctcaagtgcgtctcggtcacaatatactgttccgtcaggtgcgtgacaatccggggctcatcatggagggtgctttaggcactaacaagcttagatctatggtgtcgatcacccttgattggatcgacccccgtaatgtacccagactcactaactgggaaatgcctttgcaggagg

SEQ ID NO: 6 - DNA, 382 bp, Betacoronavirus
gatttaagtgaatagcttggctatctcacttcccctcgttctcttgcagaactttgattttaacgaacttaaataaaagccctgttgtttagcgtatcgttgcacttgtctggtgggattgtggcattaatttgcctgctcatctaggcagtggacatatgctcaacactgggtataattctaattgaatactatttttcagttagagcgtcgtgtctcttgtacgtctcggtcacaatacacggtttcgtccggtgcgtggcaattcggggcacatcatgtctttcgtggctggtgtgaccgcgcaaggtgcgcgcggtacgtatcgagcagcgctcaactctgaaaaacatcaagaccatgtgtctctaactgtgccact

SEQ ID NO: 7 - DNA, 338 bp, Betacoronavirus
ttaagcttcggcttgttgcataggaccggaaaggtactatctaccctaactcttgtagttagactctctaaacgaactttaaaactggttgtgtccttcagtagtctgtatggccattggaggcacaccggtaattatcaaatactaagaagattcatagtacatccttgtctagcttttggttggcagtgagcctacggtttcgtccgtgtcgctcacaattatccacacagtaggtttcgtccgctgtggttgagttgctagtccgttgctgtttcgtcagccatctacaactcgacaccatgtgtagcaaggggtatgaactgaatcagaaaact